(19) United States
(12) Patent Application Publication
(10) Pub. No.: US 2008/0268436 A1
(43) Pub. Date: Oct. 30, 2008

(54) Schizophrenia, Schizoaffective Disorder and Bipolar Disorder Susceptibility Gene Mutation and Applications to Their Diagnosis and Treatment

(76) Inventors: Jubao Duan, Skokie, IL (US); Raymond Crowe, Iowa City, IA (US); Maria Martinez, Evanston, IL (US)

Correspondence Address: JACOBSON HOLMAN PLLC, 400 SEVENTH STREET N.W., SUITE 600, WASHINGTON, DC, 20004, US

(21) Appl. No.: 11/660,168

(22) PCT Filed: Aug. 19, 2005
(86) PCT No.: PCT/US05/29541

§ 371 Date: Feb. 26, 2008

Related U.S. Application Data
(60) Provisional application No. 60/602,853, filed on Aug. 20, 2004.

(51) Int. Cl. C12Q 1/68; C07H 21/04

(52) U.S. Cl. 435/6; 536/23.1; 536/24.33

(57) ABSTRACT

The present invention provides the identification of a number of SNPs that are associated schizophrenia, schizoaffective disorder, bipolar disorder and related mental disorders which were found to be strongly linked to individuals with the disease. The invention provides SNP locations on human chromosome 6, as well as methods of making PCR primers and assays for detecting the SNPs in tested individuals.

### BACKGROUND OF THE INVENTION

1. Field of Invention

The present invention relates to identifying a gene that codes for a receptor as being associated with schizophrenia and schizoaffective disorder known as TRAR4, and its use in the diagnosis and screening of therapeutic agents useful in the treatment of the disease.

2. Description of Prior Art

Schizophrenia (along with the closely related schizoaffective disorder) is a frequently chronic and devastating brain disorder that affects about 1% of the population worldwide (Jablensky et al. 1992). Typically it presents in adolescence or young adulthood and is characterized by major disruptions of thinking (delusions, disorganization), perception (hallucinations), mood, and behavior (Gottesman and Shields 1982). Schizophrenia and schizoaffective disorder are strongly familial, with a heritability of about 80%, but its etiology is hypothesized to involve both genetic and environmental factors (Sanders and Gejman 2001). Recently, encouraging evidence for several genes potentially involved in the etiology of schizophrenia has been reported, namely dysbindin (DTNBP1) (Straub et al. 2002; Schwab et al. 2003), neuregulin 1 (NRG1) (Stefansson et al. 2002; Stefansson et al. 2003; Williams et al. 2003), proline dehydrogenase (oxidase) 1 (PRODH) (Jacquet et al. 2002; Liu et al. 2002), catechol-O-methyltransferase (COMT) (Li et al. 2000; Egan et al. 2001; Shifman et al. 2002), regulator of G-protein signaling 4 (RGS4) (Chowdari et al. 2002; Morris et al. 2004; Williams et al. 2004), D-amino acid oxidase activator (DAOA, previously called G72) (Chumakov et al. 2002; Schumacher et al. 2004), and D-amino-acid oxidase (DAO) (Chumakov et al. 2002; Schumacher et al. 2004). Most of the aforementioned genes are positional candidates with likely involvement with dopaminergic or NMDA brain mechanisms.

We have previously reported linkage of schizophrenia to chromosome 6q13-q26 (SCZD5; MIM 603175) (Cao et al. 1997), which since then accumulated wide, although not universal, support from converging evidence from a number of studies. In the first report of linkage to 6q (Cao et al. 1997) support for linkage was observed from D6S301 (located at 111 cM) to D6S305 (located at 170 cM), which was confirmed in subsequent studies (Kaufmann et al. 1998; Martinez et al. 1999; Levinson et al. 2000). All the families used in the studies underlying this patent application are from three datasets, which we call NIMH-IRP (National Institute of Mental Health—Intramural Research Program), NIMH-GI (NIMH-Genetics Initiative), and AU/US (Australia/United States), that previously have been shown to yield evidence for linkage to chromosome 6q13-q26 (SCZD5; MIM 603175) (Cao et al. 1997; Martinez et al. 1999; Levinson et al. 2000). Analysis of 12 microsatellite markers on a 50 cM 6q region were reported in (Martinez et al. 1999); nonparametric ASP methods yielded P-values of 0.00018, 0.00095 and 0.013 for the NIMH-IRP, NIMH GI, and AU/US datasets, respectively. In a Palestinian/Israeli pedigree sample (Lerer et al. 2003), the linkage peak with an NPL of 4.61 was at D6S292 (˜137 cM), and ˜131 to ˜144 cM contained the 1 NPL decrease portion of the linkage peak. Furthermore, another group found evidence for linkage in a Swedish pedigree from ˜170 cM to ˜180 cM (Lindholm et al. 2001). Whether these different results are better explained by the presence of more than one schizophrenia susceptibility gene in 6q or reflect typical peak variability in complex disorders (Hauser and Boehnke 1997; Hsueh et al. 2001), is currently unknown. Recently, bipolar disorder was reported to map to 6q with one study yielding a maximum LOD of 2.2 at 113 cM near D6S1021 (Dick et al. 2003b; Dick et al. 2003a) and another study reporting a maximum LOD of 3.56 at ˜124 to ˜126 cM near D6S1639 (Middleton et al. 2004), opening the prospect that a common gene for schizophrenia and bipolar disorder (and the intermediate, schizoaffective disorder) may be located in 6q.

We were interested in a MOXD1-STX7-TRARs gene cluster at 6q23.2 (132.8 cM) with prime candidates for schizophrenia (FIG. 1), harboring MOXD1 (monooxygenase, dopamine-β-hydroxylase-like 1) (Chambers et al. 1998), STX7 (syntaxin 7) (Wang et al. 1997), and all known human trace amine receptors (TRARs), namely, TRAR1, TRAR3, TRAR4, TRAR5, PNR (putative neurotransmitter receptor) (Zeng et al. 1998; Borowsky et al. 2001; Bunzow et al. 2001; Lee et al. 2001), and three TRAR pseudogenes (TRAR2 and G-protein coupled receptors, GPR57 and GPR58) (Liu et al. 1998; Borowsky et al. 2001; Bunzow et al. 2001; Lee et al. 2001).

Trace amines (TAs) are endogenous amine compounds chemically similar to classical biogenic amines such as dopamine, norepinephrine, serotonin, and histamine. Abnormalities involving the classical biogenic amines are the basis for a variety of biological hypotheses for a wide variety of disorders, including dystonias, Parkinson's disease, schizophrenia, drug addiction, and mood disorders. In mammals, TAs are present at low levels with no apparent dedicated synapses, but blockade of amine degradation leads to significant accumulations of trace amines suggesting high synthesis and turnover, as recently reviewed (Premont et al. 2001). TAs in mammals include tyramine (TYR), tryptamine, β-phenylethylamine (β-PEA), and octopamine (OCT) (Branchek and Blackburn 2003), and are all synthesized from amino acid precursors by the aromatic amino acid decarboxylase.

TAs were thought to be “false transmitters,” which displace classical biogenic amines from their storage and act on transporters in a similar fashion to the amphetamine (Parker and Cubeddu 1986), but the identification of brain receptors specific to TAs indicates that they also have effects of their own (Borowsky et al. 2001). This might explain the fact that although TYR, β-PEA, OCT, and amphetamine require the integrity of vesicular stores of dopamine if displacement of dopamine were their only mechanism of action, they (except OCT) are still active when dopamine is depleted (Baud et al. 1985). TRARs bind amphetamine, MDMA (3,4-methylenedioxymethamphetamine; “ecstasy”), and LSD (D-lysergic acid diethylamide) with high affinity. This suggests a direct link of TRARs with mechanisms of psychosis because the administration of amphetamine can induce a schizophrenia-like psychosis (Connell 1958; Snyder et al. 1967; Angrist et al. 1974; Laruelle and Abi-Dargham 1999) and psychedelic experiences induced by LSD can have remarkable similarity to schizophrenia (Vardy and Kay 1983; Gouzoulis et al. 1994). Furthermore, LSD can induce habituation deficits (the normal decrease in response magnitude to repeated stimuli over time), which are similar to those exhibited by schizophrenic patients (Geyer and Braff 1987; Braff et al. 1992).

MOXD1 is a homologue of dopamine-β-hydroxylase potentially involved with the biosynthesis of norepinephrine from dopamine (Chambers et al. 1998). Syntaxin 7 (STX7) is a critical component of the synaptic protein complex SNARE (receptor for soluble N-ethylmaleimide-non-sensitive factor attachment proteins), which is involved in NMDA (N-methyl D-aspartate) and dopaminergic receptor function (Pei et al. 2004) and whose dysfunction has been suggested in schizophrenia (Honer et al. 2002). Specifically, syntaxins mediate vesicle fusion in vesicular transport processes (Teng et al. 2001). We investigated DNA polymorphisms in the MOXD1-STX7-TRARs cluster with schizophrenia using family-based association methods and present preliminary evidence of association between TRAR4 and schizophrenia.

There are two reports suggesting linkage of the same 6q chromosomal area to bipolar disorder raise the possibility that TRAR4 might be involved in the pathophysiology of both schizophrenia and bipolar disorder (and the intermediate, schizoaffective disorder), and there is a precedent for a gene potentially involved in both disorders (Chumakov et al. 2002; Hattori et al. 2003; Chen et al. 2004; Schumacher et al. 2004).

### SUMMARY OF THE INVENTION

Molecular genetic studies of schizophrenia have found several replicated linkages to various chromosomal regions (Owen et al. 2004), and association studies have recently pointed to several genes at some of those linkage regions with independent confirmations, including NRG1 at 8p21-p12 (Stefansson et al. 2002; Stefansson et al. 2003;

Williams et al. 2003), DTNBP1 at 6p22.3 (Straub et al. 2002; Schwab et al. 2003), COMT at 22q11.21 (Li et al. 2000; Egan et al. 2001; Shifman et al. 2002), RGS4 at 1q23.3 (Chowdari et al. 2002; Morris et al. 2004; Williams et al. 2004) and DAOA at 13q33.2 (Chumakov et al. 2002; Schumacher et al. 2004). Although non-replications have been reported, it would be extremely unlikely that all the aforementioned results will end as false positives. We now propose that TRAR4 is also a susceptibility gene for schizophrenia.

The identification of TRAR4 as a susceptibility gene for schizophrenia, which is consistent with human and animal models of toxic psychosis and in agreement with the expression pattern of TRAR4 (expressed in frontal cortex, amygdala, and hippocampus), appears to substantiate the dopaminergic hypothesis of schizophrenia, but the exact mechanisms of disease mediated by TRAR4 remain to be elucidated.

Several linkage studies across multiple population groups provide convergent support for chromosome 6q13-q26 as containing a susceptibility locus for schizophrenia, and more recently for bipolar disorder. We genotyped 192 European and African American (AA) schizophrenia pedigrees (these pedigrees—in particular the NIMH-IRP sample—have many cases with schizoaffective disorder, major recurrent depression and other bipolar spectrum disorder conditions, though our study only counted schizophrenia and schizoaffective disorder as “affected”) from samples that previously showed linkage evidence to 6q13-q26, focusing on the MOXD1-STX7-TRARs gene cluster at 6q23.2, which contains a number of prime candidate genes for schizophrenia. Thirty-one screening SNPs were selected, providing a minimum coverage of at least one SNP per 20 kb. The association observed with rs4305745 (P=0.0014) within the TPAR4 (trace amine receptor 4) gene remained significant after correction for multiple testing. Evidence for association was proportionally stronger in the smaller AA sub-sample.

Through database searching and sequencing genomic DNA in a thirty probands sub-sample, we obtained a high-density map of twenty-three SNPs spanning 21.6 kb of this gene. Single SNP and also haplotype analyses revealed that rs4305745, and/or two other polymorphisms in perfect linkage disequilibrium (LD) with rs4305745, appear to be the most likely variants underlying the association of the TRAR4 region with schizophrenia. Comparative genomic analyses further revealed that rs4305745, and/or the associated polymorphisms in complete LD, could possibly affect gene expression. Moreover, RT-PCR studies of various human tissues including brain confirm that TRAR4 is preferentially expressed in those brain regions implicated in the pathophysiology of schizophrenia.

We now show that TRAR4, a gene that belongs to the trace amine receptor family contributes to susceptibility to schizophrenia in three data sets with evidence of genetic linkage to 6q. Furthermore, the TRARs gene cluster at chromosome 6q23 is contained within a wide area of linkage detected in multiple other clinical samples (Bailer et al. 2000; Levinson et al. 2000; Lindholm et al. 2001; Lerer et al. 2003; Lewis et al. 2003). The linkage evidence for schizophrenia in 6q is not population specific as it has been gathered from multiple population groups: African Americans, European Ancestry; and Jews and Arabs from Israel. However, the evidence for association of TRAR4 in our samples, although present in EA and in AA, appears higher in AA.

It is therefore an object of the invention to identify SNPs associated with Schizophrenia, schizoaffective disorder, bipolar disorder and related mental disorders.

It is another object of the invention to develop an assay in which the SNPs associated with schizophrenia, schizoaffective disorder, bipolar disorder and related mental disorders can be identified.

It is a further object of the invention to develop a PCR based assay that can identify SNPs associated with schizophrenia, schizoaffective disorder, bipolar disorder and related mental disorders on human chromosome 6.

It is also an object of the present invention to create a method for predicting a risk of an individual to human schizophrenia, schizoaffective disorder, bipolar disorder and related mental disorders, said method comprising amplifying genomic DNA of said individual using oligonucleotide primers to human chromosome 6 to obtain an amplified PCR product, identifying the nucleotides present at the polymorphic sites at nucleotides 132,874,282, 132,874,294 and 132,874,335 of human chromosome 6 (UCSC Map Position, version of July 2003), and predicting the risk of the individual to schizophrenia, schizoaffective disorder, bipolar disorder and related mental disorders based upon the haplotype present at the polymorphic sites at nucleotides 132,874,282, 132,874,294 and 132,874,335 of human chromosome 6, wherein a G at position 132,874,282 human chromosome 6, or a deletion at position 132,874,294 of human chromosome 6, or a G at position 132,874,335 of human chromosome 6 haplotype is indicative of an increased risk of developing schizophrenia, schizoaffective disorder, bipolar disorder and related mental disorders, and wherein an A at position 132,874,282 human chromosome 6, or an A at position 132,874,294 of human chromosome 6, or an A at position 132,874,335 of human chromosome 6 haplotype is indicative of a decreased risk of developing affected phenotypes. Our results also open the possibility that allelic heterogeneity for bipolar disorder and other psychiatric disorders will be found.

It is also an object of the invention to provide a diagnostic kit for detection of schizophrenia, schizoaffective disorder, bipolar disorder and related mental disorders associated SNP haplotypes (A/G at 132,874,282 position, an A/−deletion at 132,874,282 position or A/G at 132,874,335 position) comprising at least one primer selected from the group consisting of SEQ ID NOS: 27-270.

These and other objects of the invention, as well as many of the attendant advantages thereof, will become more readily apparent when reference is made to the following detailed description of the preferred embodiments.

### BRIEF DESCRIPTION OF THE DRAWINGS

FIG. 1 shows the genomic structure of the 6q23.2 gene cluster and the association mapping of the initial screening. The genomic positions are based on the UCSC July 2003 assembly of the human genome. (a) The relative position of the 6q23.2 gene cluster to the peak markers from various linkage studies. D6S424 (Cao et al. 1997; Martinez et al. 1999), D6S416 (Cao et al. 1997), D6S292 (Lerer et al. 2003) and D6S264 (Lindholm et al. 2001). (b) Genes in the 6q23.2 gene cluster. (c) The -log transformation of the FBAT P-value for the 31 SNP markers analyzed for the initial association screening. Each data point of the markers points to its relative position in the gene cluster shown in (b). The most significant marker is rs4305745 with a P-value of 0.0014; see FIG. 5, (supplementary table 5 (FIG. 9)) for FBAT P-values and other detailed information for all the initially selected markers, and table 1 for the single marker association results for all the additional TRAR4 markers examined in the dense mapping effort.

FIG. 2 shows pairwise LD in 192 founders for the TRAR4 region. (a) Relative physical position of 23 markers in TRAR4 region (shown in Table 1). ss28447862, ss28447876, ss28447865, rs8192624, and ss28447866 were excluded in the following LD measurements because of their low minor allele frequencies. (b) LD pattern in AA (left panel) and EA (right panel). The graph was generated by GOLD (Abecasis and Cookson 2000). In each LD pattern, D′ value (upper left diagonal) and P-values (lower right diagonal, converted to log P-values) are calculated from the program ldmax of the GOLD package (Abecasis and Cookson 2000).

FIG. 3 illustrates the expression pattern of TRAR4 in human tissues. (a) TRAR4 expression pattern in various human brain regions. Lane 1 is a 100 bp molecular weight standard ladder (Promega). Lanes 2-13 are human brain, human fetal brain, cerebellum, fetal liver, placental, spinal cord, control (no reverse-transcriptase added), basal ganglia, frontal cortex, substantia nigra, amygdala, and hippocampus. RT-PCR from total RNAs were presented, β-actin was used as internal control. (b) Quantatitive real-time PCR determined the relative abundance of the TRAR4 transcript in various human brain regions. (c) Comparison of gene expression of TRAR4 with TRAR1. Samples S1-S6 in (b) and (c) are basal ganglia, frontal cortex, substantia nigra, amygdala, hippocampus and cerebellum.

FIG. 4 (Supplementary FIG. 1) shows conserved non-coding regions defined by VISTA (Couronne et al. 2003) and the relative position to associated markers. (a) TRAR4 3′-flanking conserved regions generated by comparing human genome with mouse, rat, and chimpanzee genomes (upper, middle, and lower plots, respectively). The region with sequence similarity reaching 70% is defined as a conserved region and such peak areas are shaded pink. (b) Transformed -log FBAT P-values of TRAR4 SNPs versus their relative genomic positions. (a) and (b) are aligned according to the genomic position (UCSC July 2003 genome draft). (c) The local genomic sequence alignment (human-mouse) around rs4305745 and two other polymorphisms (ss28447873 and rs7452939) in perfect LD with rs4305745.

FIG. 5 (Supplementary Table 1) shows data from genotyping a total of 827 individuals from 192 families (67 NIMH-IRP, 69 NIMH-GI, 56 AU/US).

FIG. 6A-6D (Supplementary Table 2) shows the nucleotide sequences for the PCR primers, the FP-TDI and TaqMan probes, and related information for each marker identified in the study.

FIG. 7 (Supplementary Table 3) identifies the primer sequences for TRAR4 amplicons.

FIG. 8 (Supplementary Table 4) shows the results of linkage analyses of alleles sharing with individual SNPs from the MOXD1-STX7-TRARs genes cluster.

FIG. 9 (Supplementary Table 5) presents data showing SNP markers of initial screening and FBAT analysis.

FIG. 10 (Supplementary Table 6) identifies the mutations detected in TRAR4 from 30 schizophrenic patients.

FIG. 11 (Supplementary Table 7) shows the single marker association via FBAT for all markers with ten or more informative families in the sample.

FIG. 12 (Supplementary Table 8) provides a table comparing the coding variants detected in AA schizophrenia probands and AA controls.

FIG. 13 (Supplementary Table 9) shows the two marker haplotype association analysis for TRAR4.

### DETAILED DESCRIPTION AND PREFERRED EMBODIMENTS

In describing a preferred embodiment of the invention specific terminology will be resorted to for the sake of clarity. However, the invention is not intended to be limited to the specific terms so selected, and it is to be understood that each specific term includes all technical equivalents which operate in a similar manner to accomplish a similar purpose.

### Subjects and Phenotyping

Three samples were studied, which we call the NIMH-IRP, NIMH-GI, and AU/US collections. Ascertainment of the NIMH-IRP sample was described initially (Gershon et al. 1988), and the full sample from which the present sample of 67 pedigrees was drawn was described later (Cao et al. 1997; Gejman et al. 2001).

The collection of the NIMH-GI sample was described in a report of a genome scan of 71 pedigrees (Cloninger et al. 1998), and additional NIMH-GI families were subsequently included in the repository-based dataset (see electronic-database information section); 69 pedigrees were drawn for the present analysis and two previous ones (Cao et al. 1997; Martinez et al. 1999).

The AU/US sample was described initially in a report of a genome scan of 43 pedigrees (Levinson et al. 1998); full or partial trios for the present study were from 56 of the 71 pedigrees in the expanded sample utilized in linkage fine-mapping studies (Mowry et al. 2000) and in additional analyses of this dataset (Martinez et al. 1999; Levinson et al. 2000).

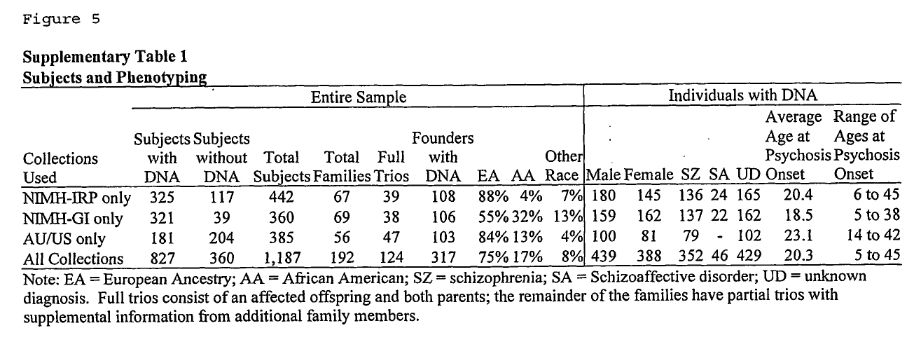

For the present study we genotyped a total of 827 individuals from 192 families (67 NIMH-IRP, 69 NIMH-GI, 56 AU/US). Details are provided in supplementary table 1 (FIG. 5) and in the electronic database information section. Individuals were diagnosed as affected with schizophrenia (the majority of the affected subjects) or schizoaffective disorder via the criteria of the Diagnostic and Statistical Manual of Mental Disorders, third edition, revised (DSM-IIIR) (APA 1987). The Institutional Review Board of the Evanston Northwestern Healthcare Research Institute approved this study.

### SNP Selection and Genotyping

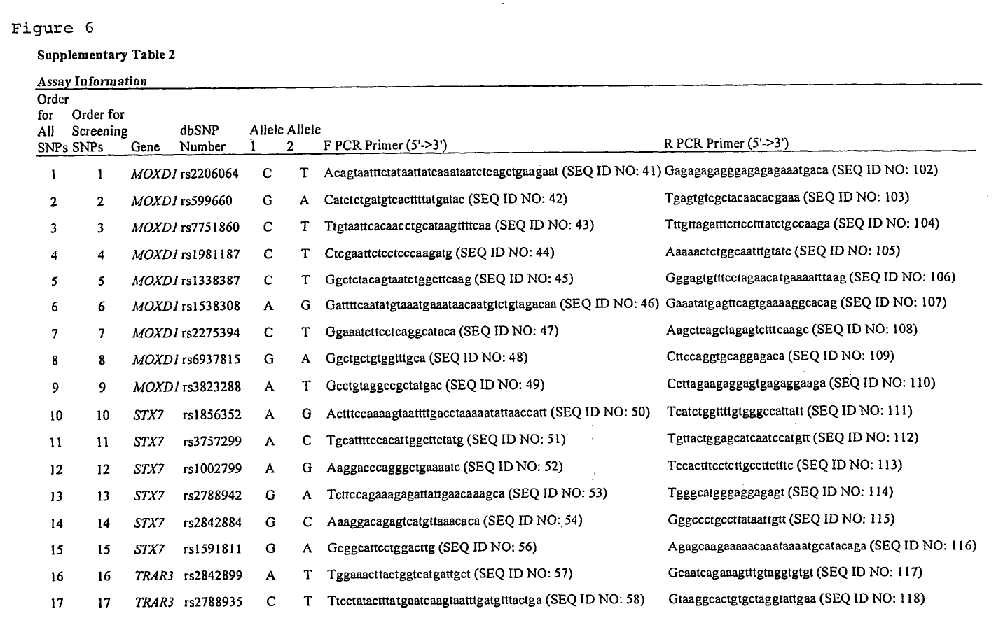

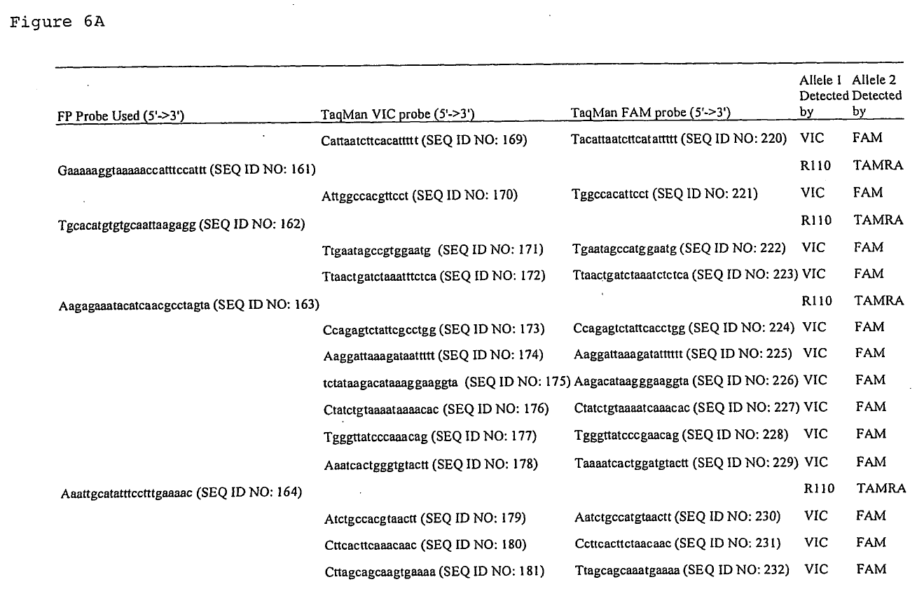

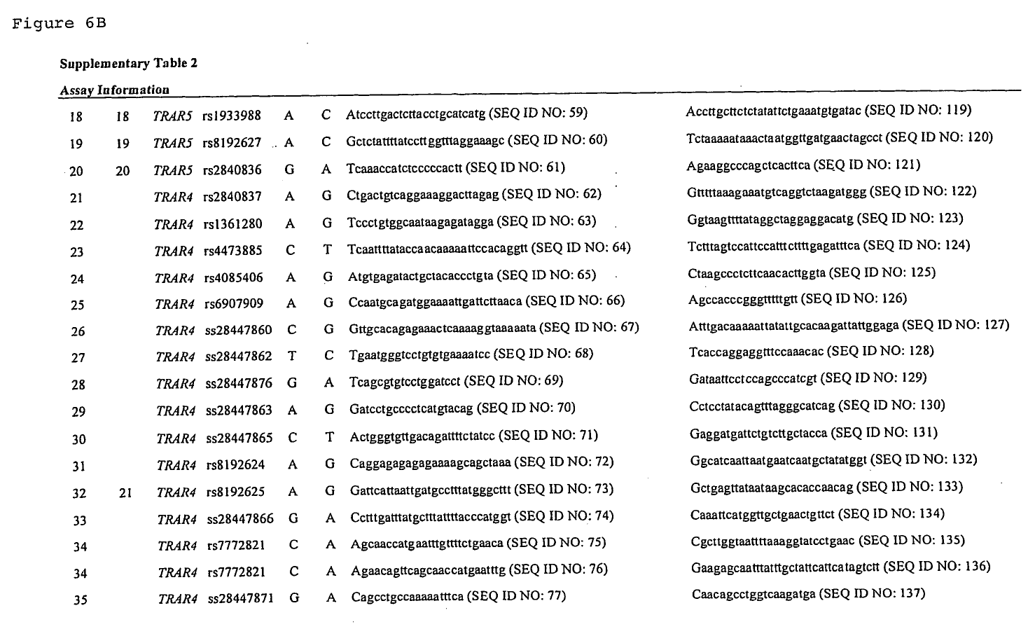

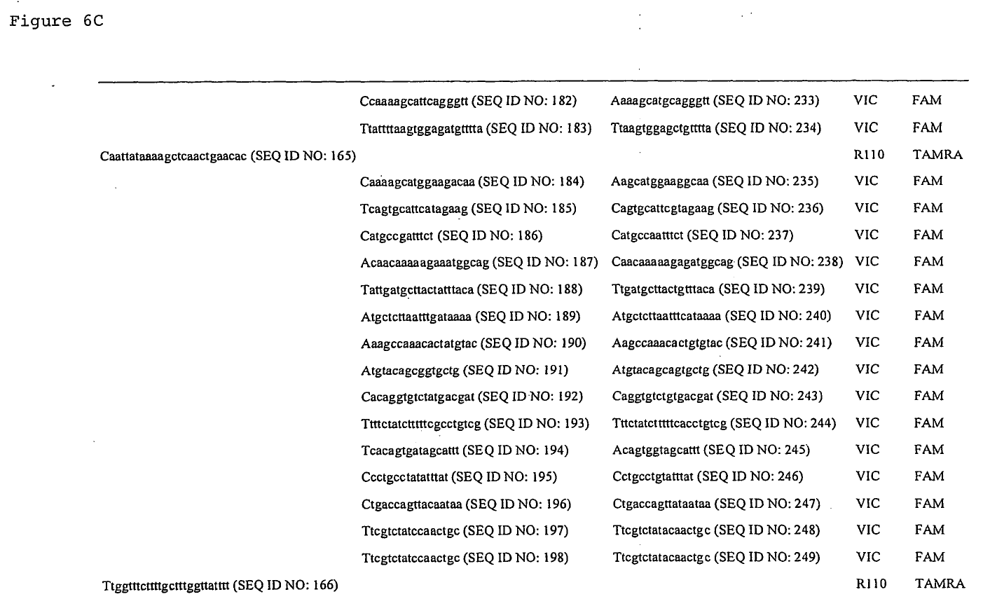

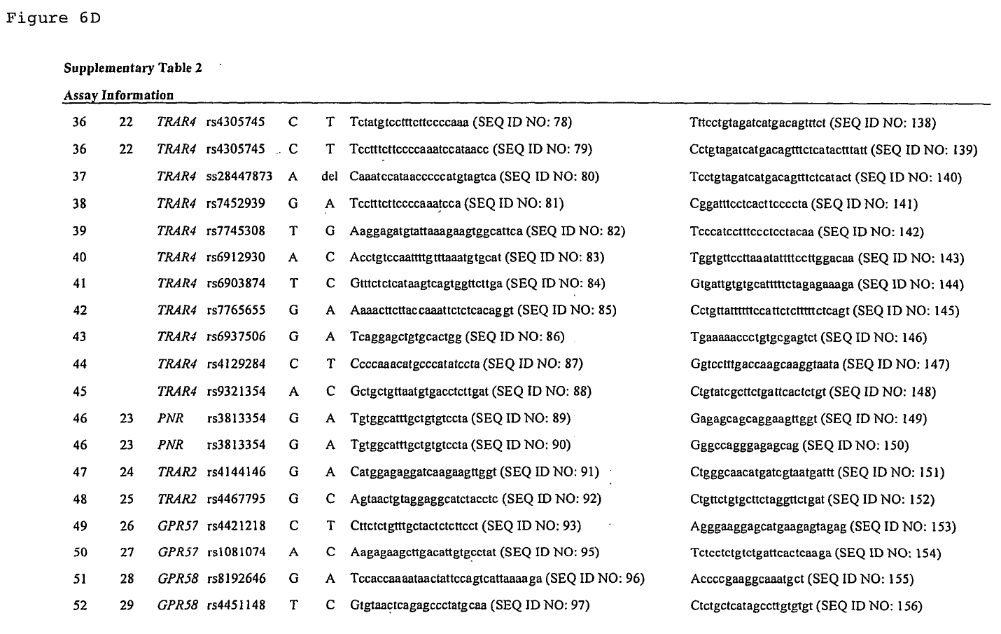

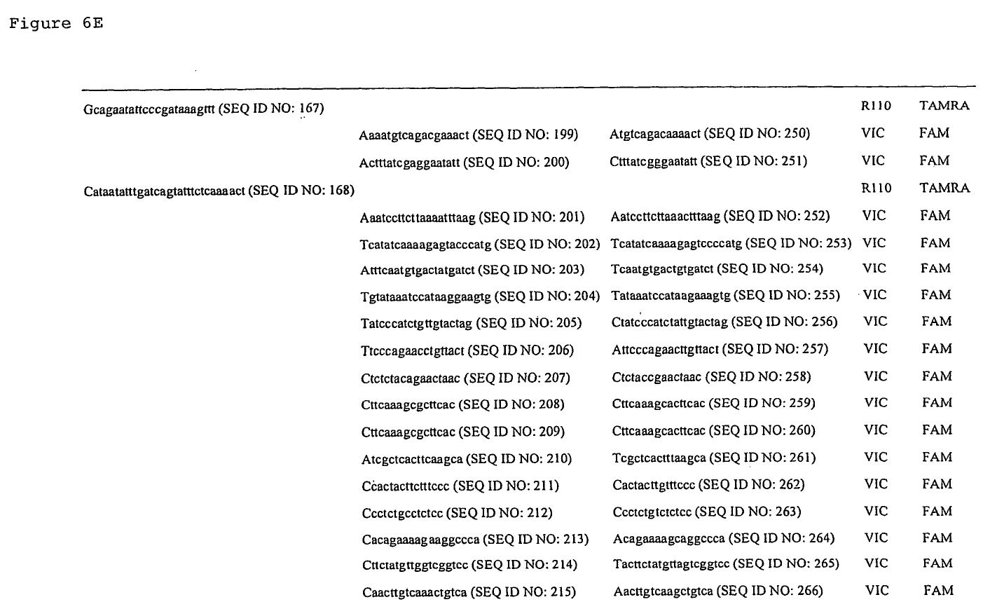

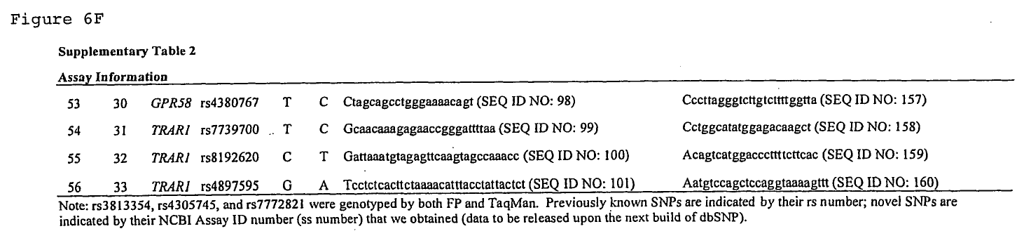

SNPs were selected from public databases with the help of a bioinformatics tool, SNPper (Riva and Kohane 2002), and novel TRAR4 SNPs were identified by direct sequencing. The DNA samples were genotyped using two methods: (1) template-directed dye-terminator incorporation with fluorescence-polarization detection (FP-TDI) (Chen et al. 1999) or (2) the TaqMan assay developed by Applied Biosystems (ABI). For the FP-TDI assays, briefly, after PCR amplification of genomic DNA, the AcycloPrime™-FP SNP detection kit (PerkinElmer) was used for post-PCR cleanup and the single base extension reaction, and we detected FP by either an Analyst fluorescence reader (LJL Biosystems) or a Wallac-Victor3 (PerkinElmer), and FP data were converted to genotypes with the assistance of an automated genotype calling spreadsheet (Akula et al. 2002). PCR primers and probes for the FP-TDI assays were designed using Primer3 (Rozen and Skaletsky 2000). For the TaqMan assays, briefly, the genomic sequence flanking the SNP was submitted to ABI for developing an assay-by-design. Each unique TaqMan minor groove binding (MGB) allele specific probe was labeled by either a 5′-FAM or a 5′-VIC reporter dye. PCR amplification of genomic DNA was performed in a 384-well plate in an ABI Prism 7900 or a DNA Engine Tetrad 2 (MJ Research). After PCR, the allele discrimination was performed on an ABI Prism 7900 Sequencing Detection System using Sequence Detector Software (SDS) version 2.0. Standard genotype calling was converted by a customized spreadsheet. Nucleotide sequences for the PCR primers, the FP-TDI and TaqMan probes, and related information for each marker can be found in supplementary table 2 (FIGS. 6A-6D).

The average completion rate of our experiments was 96%. To empirically check for errors in the genotyping method, we compared genotypes for marker rs4305745 from different methods; a difference rate between FP-TDI and TaqMan assay was about 0.25%. Using MERLIN (Multipoint Engine for Rapid Likelihood Inference) (Abecasis et al. 2002) (with all the SNPs at once), we checked Mendelian inconsistencies, blanked them as described below (sometimes for individuals and sometimes for the family when the error could not be traced to a particular individual), and then addressed all unlikely recombinants. Genotyping errors were detected for 0.17% of genotypes (MERLIN) (95 errors out of 54,611 nonzero genotypes), including 26 Mendelian inconsistencies (0.047%) and 69 unlikely recombinants (0.12%). We did not change genotypes for unlikely recombinants unless MERLIN estimated a high probability of an individual genotype error, compared to other possible errors (see MERLIN documentation for details) and/or manual re-reading each genotype tracing or other raw genotyping output for the family/marker in question pinpointed a specific error for a particular individual. All genotype errors (all Mendelian inconsistencies or when specific errors were seen that resulted in unlikely recombinants) were blanked (zeroed) for the involved individuals, and we did not perform a second pass genotyping given our high genotyping completion rate and low genotyping error. Genotypes were read blindly of psychiatric status.

We checked Hardy-Weinberg equilibrium (HWE) on family founders (at least 200) for all 55 SNPs. Three MOXD1 SNPs were found not to be in HWE (rs2206064, rs1981187 and rs2275394, though rs2206064's HWE P-value did not remain significant after taking into account the number of markers examined for HWE). The minor allele frequency of rs2206064 was only 2%; which might explain the lack of HWE. Both rs1981187 and rs2275394 had higher frequencies of homozygotes than expected (which was not a bias introduced by cleaning—only a handful genotypes were blanked and they were not primarily heterozygous genotypes).

### Intermarker Linkage Disequilibrium Analysis

LD (linkage disequilibrium) between the SNPs was estimated with the program ldmax from the GOLD (Graphical Overview of Linkage Disequilibrium) package (Abecasis and Cookson 2000) using the genotypes from unrelated founders. Ldmax estimates haplotype frequencies from genotype data using an expectation-maximization algorithm (Excoffier and Slatkin 1995). The standard and normalized Lewontin's Disequilibrium coefficients (D, D′) are derived. Association significance is assessed from a chi-squared distribution with (n₁-1) (n₂-1) degrees of freedom (df), where n₁ and n₂ are the number of alleles at each marker locus.

### Association Analysis

To detect LD with illness, we used the transmission disequilibrium test (TDT), as implemented in the Family. Based Association Test (FBAT) program v1.5 (Laird et al. 2000; Rabinowitz and Laird 2000). The null hypothesis of interest, here, is the absence of association in presence of linkage. We thus employed the empirical-variance estimator (-e flag option in the FBAT program) to account for SNP-genotype correlations among affected siblings due to linkage. The FBAT test statistic uses a score function, Z=Sⱼ−E(Sⱼ)/Var(Sⱼ), where Sⱼ is the observed number of transmitted marker alleles j to affected offspring, and E(Sⱼ) and Var(Sⱼ) are the expected and variance values of Sⱼ under the null hypothesis. Asymptotically, Z is assumed to follow a normal distribution with a mean and a variance equal to 0 and 1, respectively. The test statistic can also be expressed as Z², which follows a chi-squared distribution with 1 df. FBAT has the ability to deal with the transmission of multi-locus haplotypes, even when phase is unknown and parental genotypes may be missing. It can use both pedigrees and nuclear families, but pedigrees are broken down into all individual nuclear families, though it only includes informative families, i.e., those contributing to the test statistic. For the analyses of the screening SNPs, alleles and haplotypes were tested for association if there were at least 10 informative families; in our data this corresponds to not testing alleles and haplotypes rarer than 3%. This restriction, however, was not used when the investigation was limited to specific subsets of families in the secondary analyses. For multi-locus association analyses, FBAT provides global P-values, which assess the significance of transmission distortion for all the tested haplotypes. In the present analyses, we limited the number of multi-locus systems tested by using a stepwise procedure, and limiting the number of multi-locus tests to the combinations including the SNP with highest single Z score value, as further detailed in the results. FBAT analyses were performed assuming an additive model for each SNP, and only one affection status model was used (affected subjects had schizophrenia or schizoaffective disorder). The additive model is expected to perform well even when the true model is non-additive.

### Linkage Analysis

Model-free linkage analyses with the MOXD1-STX7-TRARs gene cluster were carried out using the lod score test from the affected-only sharing method (Kong and Cox 1997), as implemented in the MERLIN program (Abecasis et al. 2002). The likelihood of the observed marker information among affected relatives is maximized as a function of the marker alleles sharing parameter, and is compared, through a likelihood ratio test, with the likelihood of the marker data under the null hypothesis of no linkage. The resulting distribution of the allele sharing test (T) is a χ² with 1 degree of freedom, and the statistic can also be reported as a lod score=T/2ln(10).

We performed additional analyses to account for putative genetic and/or allelic heterogeneity within our family sample by ethnic origin. Association and linkage tests were evaluated separately in the subset of AA (African American) and EA (European Ancestry) families.

### Mutation Detection

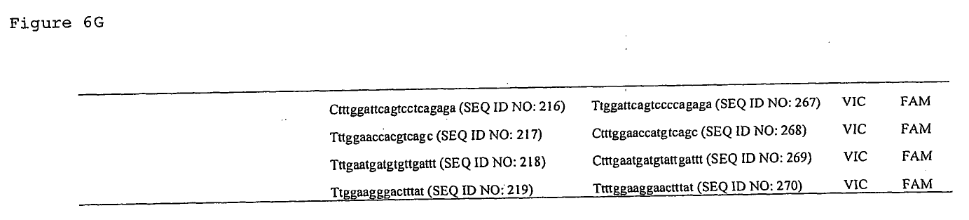

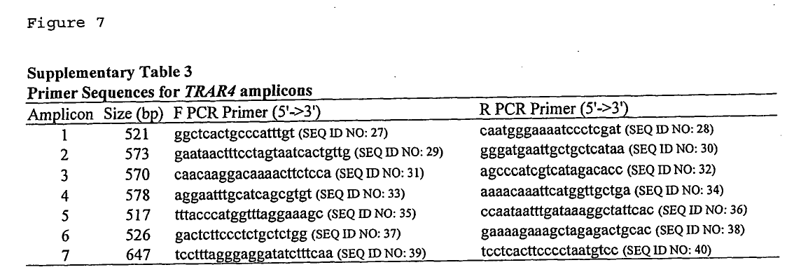

Sequencing of TRAR4 was performed on ABI 3100 genetic analyzer. Purified PCR products from various amplicons of relevant genomic DNA fragments were used as templates in sequencing reactions with the chemistry of BigDye 3.1 (ABI). PCR primers were designed by Primer 3 (Rozen and Skaletsky 2000) and were also used as sequencing primers for forward and reverse sequencing. The primer sequences and product sizes are in supplementary table 3 (FIG. 7). We used software SeqScape Ver2.1 (ABI) to assist in mutation detection, and we visually verified each mutation. The reference sequence of TRAR4 used in the analysis was from the human genome draft of the UCSC July 2003 freeze.

For the non-human primates, DNAs were extracted from peripheral blood samples of two different chimpanzees (PTR-S109 and PTR-S286) from West Africa and from tissue samples of two different lowland gorillas (GGO-S110 and GGO-S249). The forward primer of amplicon one and the reverse primer for amplicon seven were used to PCR amplify the entire DNA segment by standard methods with annealing at 60° C.; this product was then sequenced bi-directionally with the seven primer pairs detailed in supplementary table 3 (FIG. 7). PCR products were confirmed by 1.5% agarose gel electrophoresis, and purified using Micro Spin Columns (Amersham Biosciences). The purified PCR products were sequenced using the BigDye Terminator Cycle Sequencing Kit (ABI) on an ABI Prism 377/3100 DNA sequencer. Sequence data were assembled by the phred/phrap program (Ewing et al. 1998) and also were checked manually using the consed program (Gordon et al. 1998). Sequence data with both strand reads and/or high quality (more than 30 quality value) were used and deposited into the DDBJ/EMBL/GenBank International Nucleotide Sequence Database.

Accession numbers and URLs for data presented herein are as follows:

Online Mendelian Inheritance in Man (OMIM), http://www.ncbi.nlm.nih.gov/Omim/

National Institute of Mental Health (NIMH) Human Genetics Initiative for Schizophrenia, families used in this manuscript were 30101, 30103, 30104, 30106, 30108, 30110, 30111, 30112, 30113, 30114, 30116, 30119, 30122, 30123, 30124, 30126, 30127, 30128, 30130, 30131, 30132, 30133, 30134, 30136, 30140, 30142, 30146, 31102, 31107, 31108, 31109, 31114, 31115, 31118, 31119, 31129, 31130, 31135, 31137, 31139, 31155, 32108, 32109, 32200, 32201, 32202, 32203, 32204, 32205, 32206, 32209, 32211, 32212, 32217, 32218, 32303, 32304, 32306, 32307, 32309, 32310, 32311, 32312, 32313, 32315, 32319, 32320, 32402, and 32403, http://zork.wustl.edu/nimh/sz.html

University of California at Santa Cruz (UCSC) Genome Bioinformatics July 2003 assembly of the human genome, http://genome.ucsc.edu/

SNPper bioinformatics tool, http://snpper.chip.org/

SIFT bioinformatics tool, http://blocks.fhcrc.org/sift/SIFT.html PolyPhen bioinformatics tool, http://tux.embl-heidelberg.de/ramensky/

VISTA bioinformatics tool, http://www-gsd.lbl.gov/vista/

NCBI's SNP database, http://www.ncbi.nlm.nih.gov/SNP/

NCBI's Entrez search engine, http://www.ncbi.nlm.nih.gov/Entrez/

Mfold RNA structure prediction, http://www.bioinfo.rpi.edu/applications/mfold/

G protein-coupled receptors database, http://www.gpcr.org/

dbSNP, NCBI's database of “Single Nucleotide Polymorphisms”, was used to deposit the 18 novel SNPs we found, http://www.ncbi.nlm.nih.gov/SNP/

The dbSNP accession numbers are ss28447859 through ss28447876 and will become available to the public when NCBI releases the latest dbSNP build, and at that time will be incorporated into rs#'s (Reference Cluster ID #'s) as well.

dbSTS, NCBI's database of “Sequence Tagged Sites”, was used to deposit the 18 novel SNPs we found, http://www.ncbi.nlm.nih.gov/dbSTS/

The GenBank accession numbers are BV154568 through BV154585.

The DDBJ/EMBL/GenBank International Nucleotide Sequence Database was used to deposit the gorilla and chimpanzee [0052] TRAR4 region sequences, http://www.ncbi.nih.gov/Genbank/index.html

The DDBJ/EMBL/GenBank accession numbers for the chimpanzee and gorilla sequences are AB180397 through AB180400.

### RT-PCR and Real-Time PCR

Total mRNAs from various brain tissues were purchased from either BD Biosciences or Ambion. Gene expression of TRAR4 was first confirmed with general RT-PCR with primer pairs used previously for amplification of segment 4 of TRAR4 shown in supplementary table 3 (FIG. 7). In brief, total mRNA was reverse-transcribed with TaqMan Reverse Transcription Reagents (ABI), and the synthesized first-strand cDNAs were then used as templates to amplify TRAR4 with HotStart Taq polymerase (Qiagen). β-actin was used as internal control in the RT-PCR.

Reverse transcribed cDNAs were also used in real-time PCR on an ABI Prism 7900 Sequence Detection System according to the manufacturer's protocol. The TaqMan MGB probes and PCR primer pairs for gene expression assay for TRAR4, GAPD (glyceraldehyde-3-phosphate dehydrogenase), or TRAR1 were purchased as an Assay-On-Demand from ABI (Applied Biosystems; Foster City, Calif.). The relative gene expression in different brain tissues was normalized to GAPD expression by using the standard curve method as described by ABI.

### Bioinformatic Tools for Prediction of Functional Effects of Genetic Polymorphisms

SIFT

(http://blocks.fhcrc.org/sift/SIFT.html) and PolyPhen (http://tux.embl-heidelberg.de/ramensky/) were used to predict the potential functional effect of missense polymorphisms (Ramensky et al. 2002; Ng and Henikoff 2003). We used VISTA (http://www-gsd.lbl.gov/vista/) to predict the potential regulatory sequence through defining the conserved region among genomic sequences different species (Couronne et al. 2003).

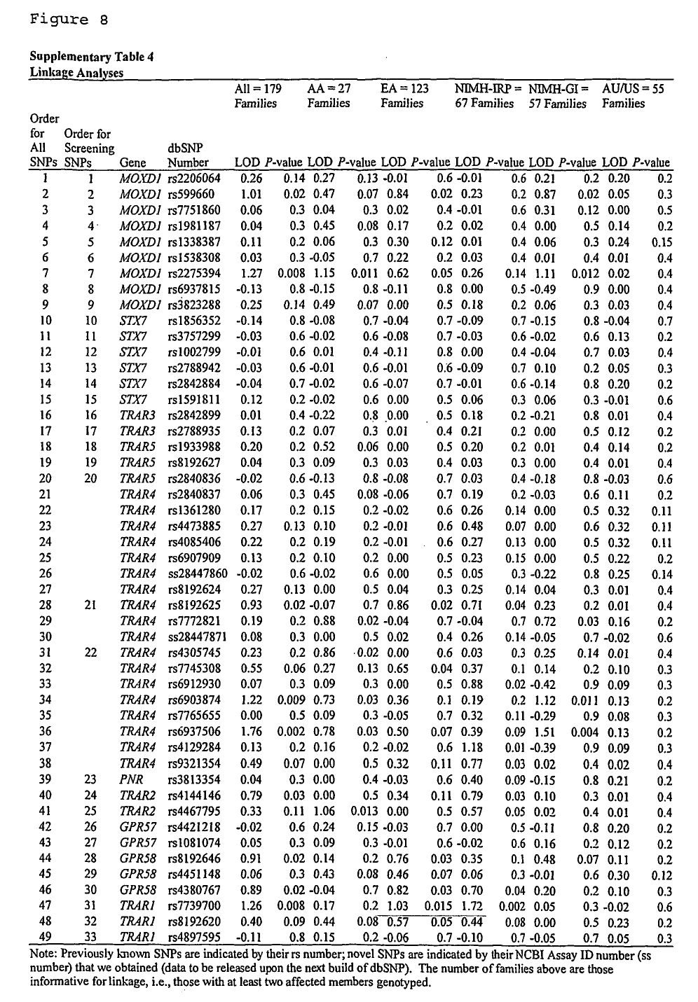

We have studied 192 families with previous evidence of linkage. Thirty-three SNPs were initially selected for study, of which thirty-one were selected for analysis in the screening experiment since two of the MOXD1 SNPs, rs2206064 and rs7751860, had minor allele frequencies resulting in less than 3% informative families. The screening SNPs spanned ˜500 kb of the MOXD1-STX7-TRARs genes cluster, a prime set of positional and pathophysiological candidates for schizophrenia. We selected at least one common SNP for each gene with a minimum coverage of ≧1 SNP per 20 kb (the screening set of SNPs). Linkage analyses confirmed the presence of excess allele sharing in this region with individual SNPs from the MOXD1-STX7-TRARs genes cluster. Nine SNPs showed linkage P-values<0.05 as seen in supplementary table 4 (FIG. 8), with the most significant one being rs6937506 (TRAR4) with a LOD=1.76, P=0.002.

The following examples will provide illustrations of the use of the invention.

### EXAMPLE 1

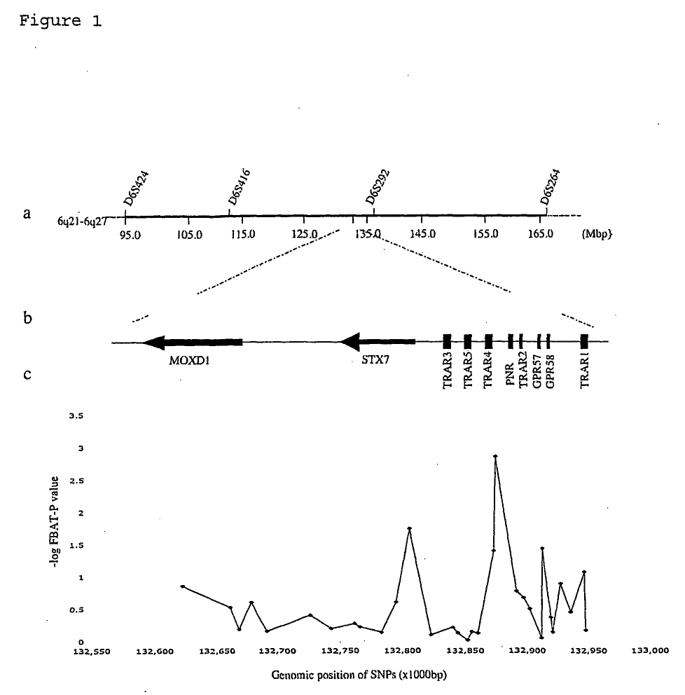

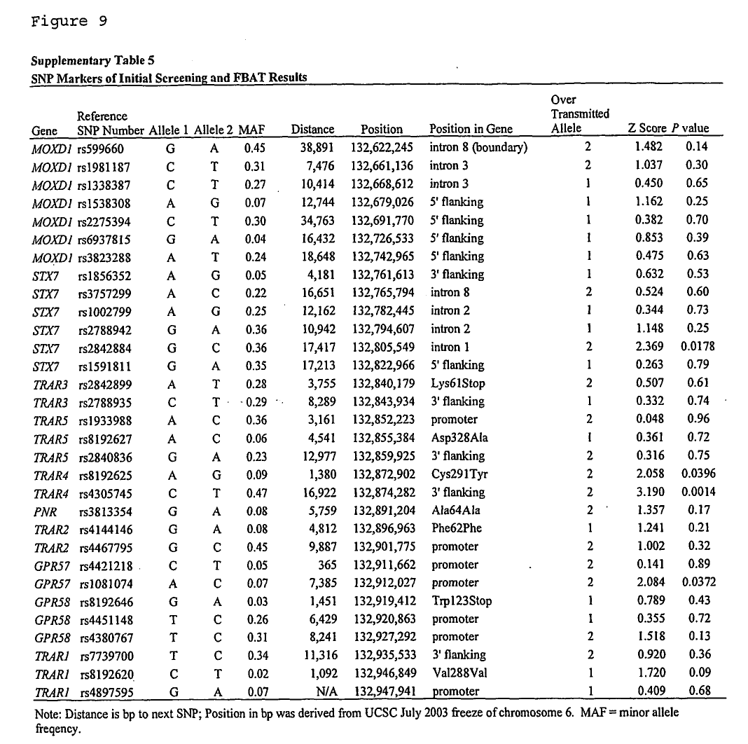

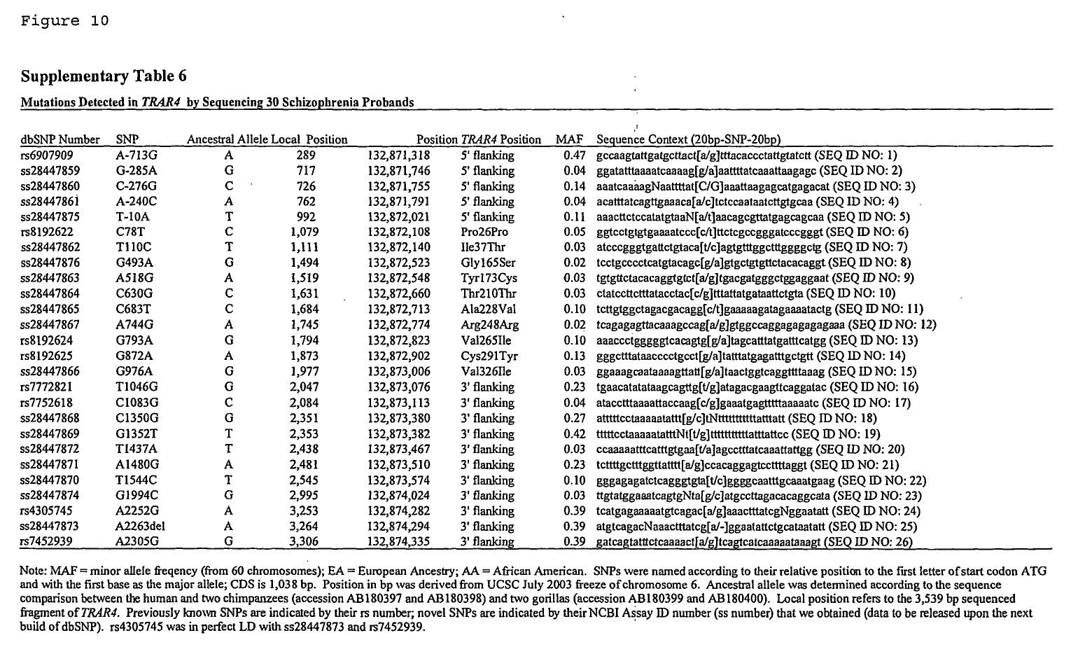

Association results are presented in table 1 (FIG. 5) and can also be seen in supplementary table 5 (FIG. 9). Of 31 screening SNPs, four SNPs spanning 106 kb, two located in TRAR4, and one each in STX7 and GPR57, yielded a P-value<0.05. The most significant one was found in TRAR4: rs4305745 (P=0.0014) (FIG. 1c), which was the only SNP that remained significant after Bonferroni correction for 31 tests. Association rs4305745 is located 1,214 bp downstream from the stop codon of TRAR4. We therefore concentrated further laboratory efforts on TRAR4. We aimed for a high-density map of>1 SNP per 2 kb by searching public SNP databases and sequencing genomic DNA. The TRAR4 gene was sequenced (˜1 kb of the 5′ region, the 1,038 bp CDS, and ˜1.5 kb of the 3′UTR, which spans rs4305745) in 30 probands selected from the NIMH-GI families: 16 European Ancestry (EA) and 14 African Americans (AA). Ten coding variants (26 total variants as seen in table 2 and supplementary table 6, FIG. 10) were found by sequencing TRAR4, including three previously found in 96 healthy EA individuals (Freudenberg-Hua et al. 2003). Five of the 7 novel variants are missense and present only in AA, shown in table 2. Twenty SNPs spanning 21.6 kb of the TRAR4 gene (>1 SNP per 2 kb) were identified for genotyping.

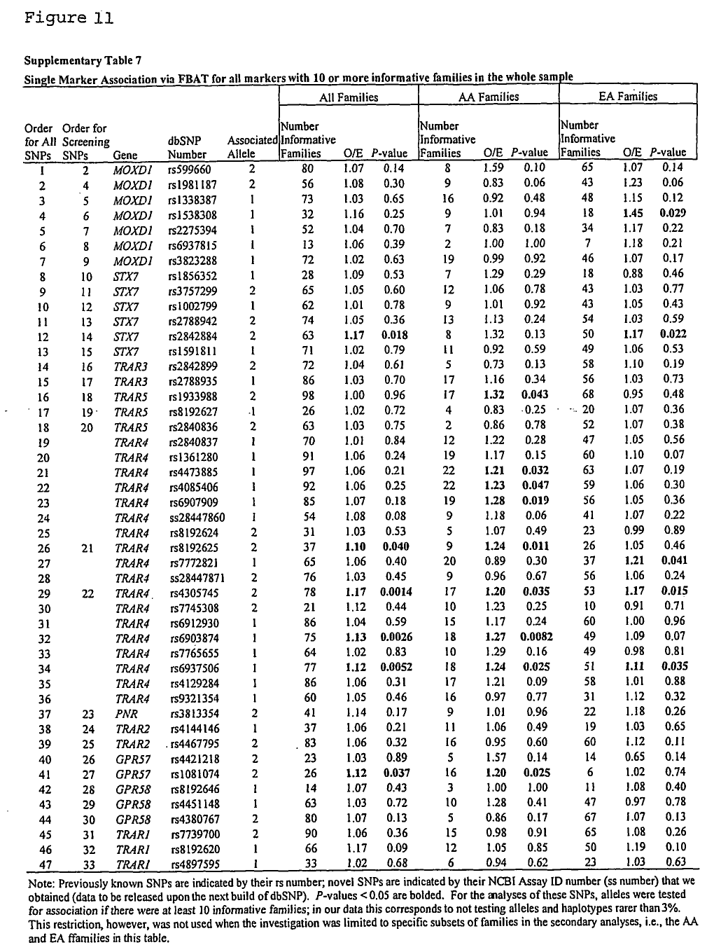

Two additional markers 3′ to rs4305745 showed association with schizophrenia in the whole sample as seen in table 1 and supplementary table 7 (FIG. 11), rs6903874 (P=0.0026) and rs6937506 (P=0.0052); and two other additional markers (ss28447873, an insertion/deletion polymorphism, and rs7452939) were found to be in perfect LD with rs4305745 (they span 53 bp), and, hence, also associated to the same degree with illness as rs4305745.

Although similar association trends in EA and AA were observed, supplementary table 7 (FIG. 11) shows that the evidence for association was proportionally stronger in the AA sub-sample. The evidence for association with rs4305745 and rs6937506 was significant in both samples (P=0.035 in AA and P=0.015 in EA for rs4305745; P=0.025 in AA and P=0.035 in EA for rs6937506), however, a cluster of three SNPs in the promoter of TRAR4 yielded significant association in the AA sub-sample: rs4473885 (P0.032), rs4085406 (P=0.047), and rs6907909 (P0.019), but not in the EA sample as seen in supplementary table 7 (FIG. 11). These differences raise the possibility of allelic heterogeneity specific to the AA population. All new missense mutations detected by sequencing were also exclusive to the AA sub-sample as seen in table 2. Two, A518G (Tyr173Cys) and C683T (Ala228Val), were predicted by either PolyPhen or SIFT (Ramensky et al. 2002; Couronne et al. 2003; Ng and Henikoff 2003) to be non-conservative, as shown in table 2.

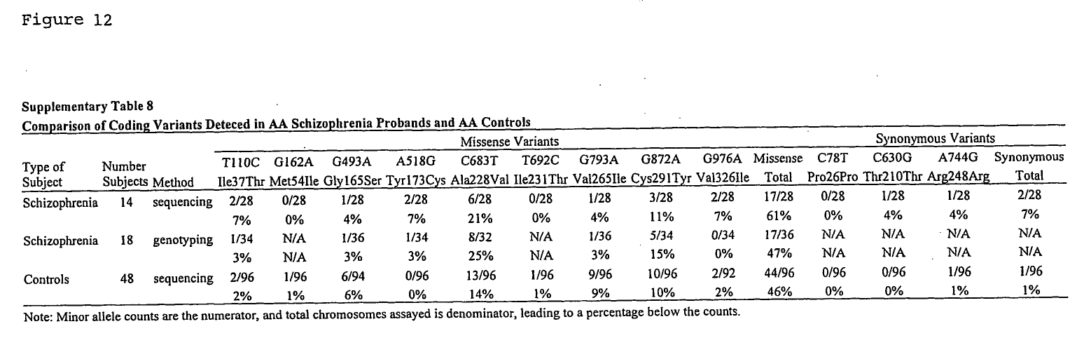

After genotyping the whole sample, it was noted that none of the newly found missense SNPs co-segregated with disease in a specific manner (data not shown). Furthermore, all the missense variants except for A518G (Tyr173Cys) were also found in a set of 48 AA subjects from the Coriell Human Variation AA DNA panel, shown in supplementary table 8 (FIG. 12); additionally, some of these missense variants were homozygous in some control individuals. Interestingly, the ratio of missense to synonymous mutations (9:3) is close to what is expected under neutral expectations, i.e., a pseudogene, which has an expected ratio close to 4:1. Indeed, in the gorilla TRAR4 has already become a pseudogene (with a nonsense mutation at codon 15: Tyr in chimpanzee and human, STOP in gorilla), though the human and chimpanzee TRAR4 versions have not yet become pseudogenes.

### EXAMPLE 2

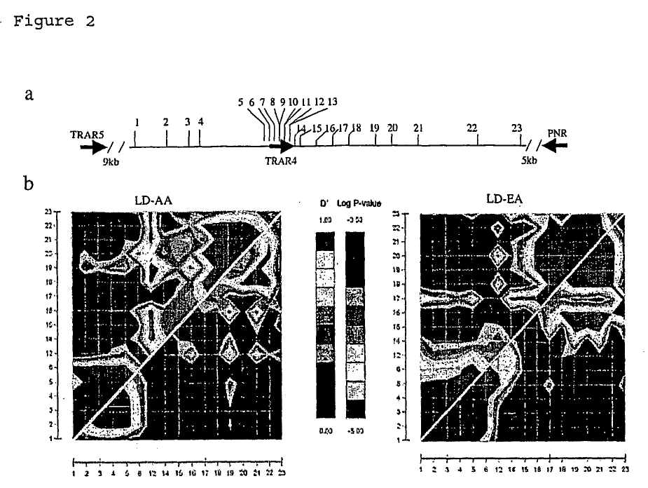

The TRAR4 region was found to have two LD blocks, depicted in FIG. 2. Association rs4305745 (marker 16 in FIG. 2) is in the LD block constituted by 3′-flanking SNPs. The pattern suggests that association for TRAR4 originates from rs4305745. None of the 5′-flanking SNPs are in LD with rs4305745, which Instead is in strong LD with markers 19 (rs6903874) and 21 (rs6937506) from the 3′ LD block (and also shows a trend with marker 12, rs8192625). The LD pattern generated from the 31 initial screening markers indicated that the whole region of MOXD1-STX7-TRARs is separated into 4 major strong LD blocks, while the TRAR4 region represented by rs4305745 is not in strong LD with any of the major LD.

### EXAMPLE 3

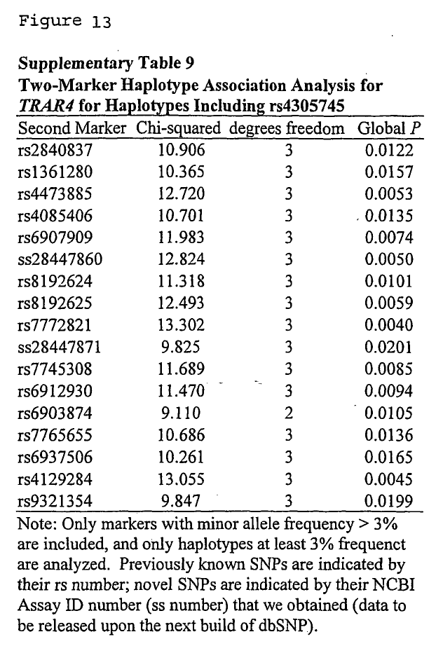

Haplotype association analyses with all TRAR4 two-locus systems were conducted (n=17, after excluding five markers with minor allele frequencies<3%) that were derived from rs4305745. This association was chosen as the anchor because it had the most significant single locus association (P=0.0014). For each such two-locus system, we derived the global χ2 value using only those haplotypes with frequencies>3%, detailed in supplementary table 9 (FIG. 13). All 17 of these two-locus systems exhibited P-values<0.05 (not corrected by multiple testing) and all harbored the “A” allele of rs4305745. None of the two-locus systems showed stronger association than rs4305745 alone as seen in supplementary table 9 (FIG. 13), and the same results were obtained with up to five multilocus systems, with each haplotype system extended stepwise to contain the most significant previous smaller haplotype (data not shown). This shows that rs4305745, and/or the other two nearby polymorphisms, ss28447873 and rs7452939, in perfect LD (further confirmed by genotyping the whole sample) with rs4305745 (supplementary table 6 (FIG. 10)), are the mutations underlying the association of the TRAR4 region with schizophrenia.

To explore the possible functional effects of associated SNPs and their haplotypes, we first defined the conserved non-coding sequence (considered as a potential functional region) by comparative genomic analysis of TRAR4 genomic sequences of human, mouse, and rat using VISTA (Couronne et al. 2003). The cluster of three polymorphisms (rs4305745, ss28447873, and rs7452939—all equally implicated as candidates by the association analysis) exhibiting the most significant association is very close to two conserved regions (sequence identity>70% among human, mouse, and rat genomes) right after the stop codon. The sequence identity immediately around this SNP (rs4305745) is about 50% as seen in supplementary FIG. 1, suggesting that this SNP and/or other polymorphisms in perfect LD with rs4305745 may ultimately affect gene expression, a hypothesis we are currently testing. RT-PCR from various brain tissues also confirmed that rs4305745 was flanked by the 3′UTR of TRAR4 (data not shown), suggesting that rs4305745 or one of its haplotypes may affect the gene expression at the post-transcriptional level. Another significant SNP, rs6903874, is also within a conserved region (supplementary FIG. 1), and it is also possible that this SNP and/or other SNPs nearby might be functional, though it is much farther from the stop codon of TRAR4 than rs4305745.

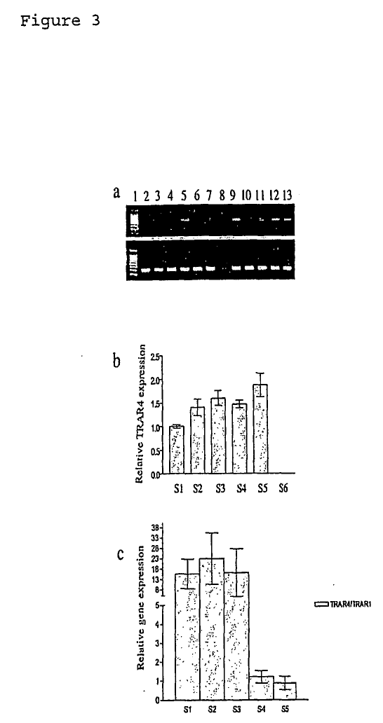

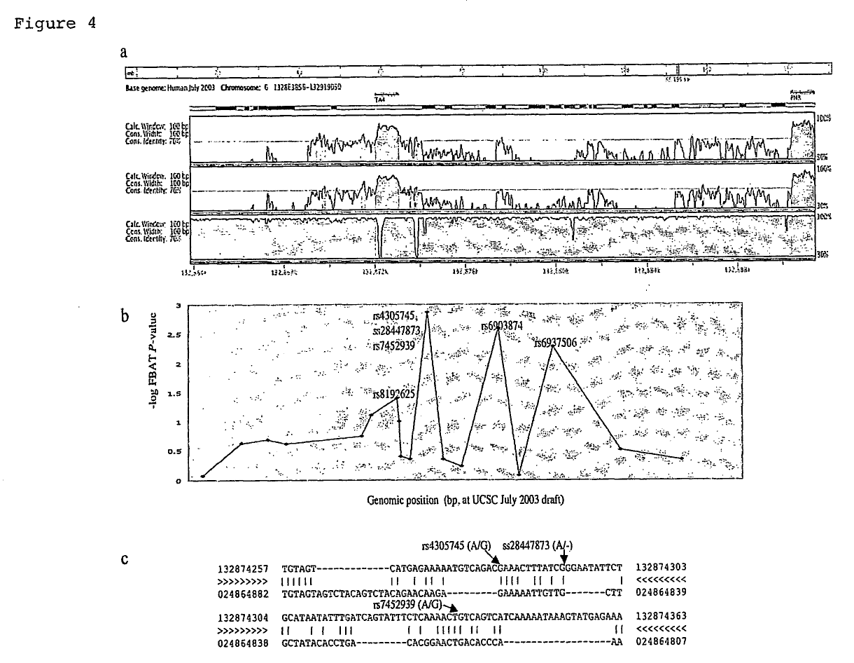

A comprehensive gene expression analysis of TRAR4 will help to elucidate its potential functional roles in the pathophysiology and pharmacology of schizophrenia. TRAR4 expression was investigated in various human tissues by RT-PCR and found that TRAR4 was expressed at low abundance in various human brain tissues as well as in human fetal liver, but not in the cerebellum or placenta as seen in FIG. 3a. A quantitative real-time PCR shown in FIG. 3b further revealed that TRAR4 has comparable levels of expression in basal ganglia, frontal cortex, substantia nigra, amygadala, and hippocampus, with highest expression in hippocampus and lowest expression in basal ganglia; these results are consistent with a previous expression study including TRAR4 (Borowsky et al. 2001). These regions have been implicated in the pathophysiology and pharmacology of schizophrenia (Grossberg 2000; Freedman 2003). The tissue distribution of TRAR4 gene expression is similar to the only well-characterized trace amine receptor, TRAR1 (Borowsky et al. 2001; Bunzow et al. 2001), however, further comparison of gene expression of TRAR4 and TRAR1 indicated that TRAR4 is overall more abundant than TRAR1, particularly in basal ganglia (˜14 fold), frontal cortex (˜21 fold), and substantia nigra (˜14 fold) (FIG. 3c), suggesting TRAR4 plays a more important role than TRAR1.

Regulatory sequence disruption can affect protein expression and cause disease (Mitchison 2001). The associated SNPs in the 3′UTR of TRAR4 may contribute to the susceptibility for the disease by affecting the gene expression at the post-transcriptional level. Our RT-PCR experiment indicated that the TRAR4 3′UTR spanned the most associated SNP rs4305745; therefore, it is possible TRAR4 gene expression was affected at the post-transcriptional level by these 3′UTR SNPs (rs4305745 and/or ss28447873 and rs7452939, two SNPs in perfect LD with rs4305745). The chimpanzee and gorilla sequencing result indicated that ancestral allele for rs4305745 is A (supplementary table 6 (FIG. 10)). Because of the perfect LD among rs4305745, ss28447873, and rs7452939, the associated allele A of rs4305745 actually represents a human haplotype of A-A-A spanning rs4305745, ss28447873, and rs7452939 (supplementary FIG. 1c; also note ancestral haplotype appears to be A-A-G as seen in supplementary table 6 (FIG. 10)). Interestingly, the predicted TRAR4 mRNA structure exhibited a significant change for the over-transmitted haplotype A-A-A compared to haplotype G-del-G, and the same structure change can be generated by allele A of rs4305745 alone (data not shown), suggesting rs4305745 is most likely the causative SNP.

We have found that the mutation rate in coding region for TRAR4 (1 mutation per 100 bp) is well above the average (1 mutation per 346 bp) (Cargill et al. 1999), and there are more missense mutations in TRAR4 than synonymous mutations, 9 versus 3, as seen in table 2 and supplementary table 8 (FIG. 12). We did not find evidence of association with schizophrenia for missense SNPs, however, some missense mutations may be pharmacologically important, particularly A518G (Tyr173Cys) located in the putative extracellular domain of the receptor and hence may affect ligand binding as seen in table 2. Besides the changes in protein structure, these missense mutations may also possibly alter the gene expression by affecting mRNA folding structures as described for dopamine D2 receptor (DRD2) (Duan et al. 2003). Actually, A518G was predicted to have a remarkable effect on TRAR4 mRNA folding as predicted in silico by mFold (Zuker et al. 1999) (data not shown). It is also notable that this missense SNP and several others were only found in AA, and 173Cys was only detected in AA schizophrenia probands.

Having described the invention, many modifications thereto will become apparent to those skilled in the art to which it pertains without deviation from the spirit of the invention as defined by the scope of the appended claims.

What is claimed is:

1. A diagnostic kit for the detection of SNP haplotypes associated with human schizophrenia, schizoaffective disorder, bipolar disorder and related mental disorders comprising at least one nucleic acid consisting of a nucleic acid selected from the group consisting of SEQ ID NO: 1-26.

2. An oligonucleotide primer consisting of a sequence selected from the group consisting of:
SEQ ID NOS: 27-270 and complements thereof.

3. A method for predicting a risk of an individual to human schizophrenia, schizoaffective disorder, bipolar disorder and related mental disorders, said method comprising:
a) amplifying genomic DNA of said individual using oligonucleotide primers to human chromosome 6 to obtain an amplified PCR product;
b) identifying the nucleotides present at the polymorphic sites at nucleotides 132,874,282, 132,874,294 and 132,874,335 of human chromosome 6 (UCSC Map Position, version of July 2003; and
c) predicting the risk of the individual to schizophrenia, schizoaffective disorder, bipolar disorder and related mental disorders based upon the haplotype present at the polymorphic sites at nucleotides 132,874,282, 132,874,294 and 132,874,335 of human chromosome 6, wherein a G at position 132,874,282 human chromosome 6, or a deletion at position 132,874,294 of human chromosome 6, or a G at position 132,874,335 of human chromosome 6 haplotype is indicative of an increased risk of developing schizophrenia, schizoaffective disorder, bipolar disorder and related mental disorders, and wherein an A at position 132,874,282 human chromosome 6, or an A at position 132,874,294 of human chromosome 6, or an A at position 132,874,335 of human chromosome 6 haplotype is indicative of a decreased risk of developing schizophrenia, schizoaffective disorder, bipolar disorder and related mental disorders.

4. The method as claimed in claim 3 wherein the primers are selected from the group consisting of:
SEQ ID NOS: 27-270 and complements thereof.

5. A diagnostic kit for detection of schizophrenia, schizoaffective disorder, bipolar disorder and related mental disorders associated SNP haplotypes (A/G at 132,874,282 position, an A/− deletion at 132,874,282 position or A/G at 132,874,335 position) comprising at least one primer selected from the group consisting of SEQ ID NOS: 27-270.

6. A method of detection of human chromosome 6 gene variants above said method comprises: (a) amplifying genomic DNA of schizophrenia, schizoaffective disorder, bipolar disorder and related mental disorders patients and normal control individuals using the primers for human chromosome 6 (SEQ ID 27-270); (b) sequencing the amplified PCR product and identifying the sequence variation computationally by comparing it with the already existing sequence of human chromosome 6; (c) screening normal control individuals and schizophrenia, schizoaffective disorder, bipolar disorder and related mental disorders patients. (d) computing the frequency of A/G haplotypes at 132,874,282 position, A/− deletion haplotypes at 132,874,282 position and A/G haplotypes at 132,874,335 position; (e) establishing the association of G (at 132,874,282 position), A/− deletion (at 132,874,282 position) and G (at 132,874,335 position) haplotypes with schizophrenia and related disease based on their frequency distribution in normals and schizophrenia, schizoaffective disorder, bipolar disorder and related mental disorders; (g) predicting the risk or susceptibility schizophrenia, schizoaffective disorder, bipolar disorder and related mental disorders based on the haplotype present at the polymorphic sites in the individual tested, G (at 132,874,282 position), a deletion (at 132,874,282 position) and a G (at 132,874,335 position) haplotypes being at high risk and A (at 132,874,282 position), A (at 132,874,282 position) and A (at 132,874,335 position) haplotypes at low risk for the disease.


SEQUENCE LISTING (272 sequences)

SEQ ID NO: 1 - DNA, 41 bp, Homo sapiens
gccaagtattgatgcttactrtttacaccctattgtatctt

SEQ ID NO: 2 - DNA, 40 bp, Homo sapiens
ggatatttaaaatcaaaagraattttatcaaattaagagc

SEQ ID NO: 3 - DNA, 40 bp, Homo sapiens
  modified_base (11): a, c, g, t, unknown or other
aaatcaaaagnaattttatsaaattaagagcatgagacat

SEQ ID NO: 4 - DNA, 40 bp, Homo sapiens
acatttatcagttgaaacamtctccaataatcttgtgcaa

SEQ ID NO: 5 - DNA, 40 bp, Homo sapiens
  modified_base (19): a, c, g, t, unknown or other
aaacttctccatatgtaanwaacagcgttatgagcagcaa

SEQ ID NO: 6 - DNA, 40 bp, Homo sapiens
ggtcctgtgtgaaaatcccyttctcgccgggatcccgggt

SEQ ID NO: 7 - DNA, 41 bp, Homo sapiens
atcccgggtgattctgtacayagtgtttggctttggggctg

SEQ ID NO: 8 - DNA, 41 bp, Homo sapiens
tcctgcccctcatgtacagcrgtgctgtgttctacacaggt

SEQ ID NO: 9 - DNA, 41 bp, Homo sapiens
tgtgttctacacaggtgtctrtgacgatgggctggaggaat

SEQ ID NO: 10 - DNA, 41 bp, Homo sapiens
ctatccttctttatacctacstttattatgataattctgta

SEQ ID NO: 11 - DNA, 41 bp, Homo sapiens
tcttgtggctagacgacaggygaaaaagatagaaaatactg

SEQ ID NO: 12 - DNA, 41 bp, Homo sapiens
tcagagagttacaaagccagrgtggccaggagagagagaaa

SEQ ID NO: 13 - DNA, 41 bp, Homo sapiens
aaaccctgggggtcacagtgrtagcatttatgatttcatgg

SEQ ID NO: 14 - DNA, 41 bp, Homo sapiens
gggctttataacccctgcctrtatttatgagatttgctgtt

SEQ ID NO: 15 - DNA, 41 bp, Homo sapiens
ggaaagcaataaaagttattrtaactggtcaggttttaaag

SEQ ID NO: 16 - DNA, 41 bp, Homo sapiens
tgaacatatataagcagttgkatagacgaagttcaggatac

SEQ ID NO: 17 - DNA, 41 bp, Homo sapiens
atacctttaaaattaccaagsgaaatgagtttttaaaaatc

SEQ ID NO: 18 - DNA, 40 bp, Homo sapiens
  modified_base (22): a, c, g, t, unknown or other
atttttcctaaaaatatttstntttttttttttatttatt

SEQ ID NO: 19 - DNA, 41 bp, Homo sapiens
  modified_base (19): a, c, g, t, unknown or other
tttttcctaaaaatatttntktttttttttttatttattcc

SEQ ID NO: 20 - DNA, 41 bp, Homo sapiens
ccaaaaatttcatttgtgaawagcctttatcaaattattgg

SEQ ID NO: 21 - DNA, 41 bp, Homo sapiens
tcttttgctttggttattttrccacaggagtccttttaggt

SEQ ID NO: 22 - DNA, 41 bp, Homo sapiens
gggagagatctcagggtgtayggggcaatttgcaaatgaag

SEQ ID NO: 23 - DNA, 41 bp, Homo sapiens
  modified_base (18): a, c, g, t, unknown or other
ttgtatggaaatcagtgntasatgccttagacacaggcata

SEQ ID NO: 24 - DNA, 41 bp, Homo sapiens
  modified_base (33): a, c, g, t, unknown or other
tcatgagaaaaatgtcagacraaactttatcgnggaatatt

SEQ ID NO: 25 - DNA, 42 bp, Homo sapiens
  modified_base (10): a, c, g, t, unknown or other
  modified_base (22): This base may or may not be present
atgtcagacnaaactttatcgaggaatattctgcataatatt

SEQ ID NO: 26 - DNA, 41 bp, Homo sapiens
gatcagtatttctcaaaactrtcagtcatcaaaaataaagt

SEQ ID NO: 27 - DNA, 18 bp, Artificial Sequence
  Description of Artificial Sequence: Synthetic primer
ggctcactgcccatttgt

SEQ ID NO: 28 - DNA, 20 bp, Artificial Sequence
  Description of Artificial Sequence: Synthetic primer
caatgggaaaatccctcgat

SEQ ID NO: 29 - DNA, 27 bp, Artificial Sequence
  Description of Artificial Sequence: Synthetic primer
gaataactttcctagtaatcactgttg

SEQ ID NO: 30 - DNA, 21 bp, Artificial Sequence
  Description of Artificial Sequence: Synthetic primer
gggatgaattgctgctcataa

SEQ ID NO: 31 - DNA, 22 bp, Artificial Sequence
  Description of Artificial Sequence: Synthetic primer
caacaaggacaaaacttctcca

SEQ ID NO: 32 - DNA, 20 bp, Artificial Sequence
  Description of Artificial Sequence: Synthetic primer
agcccatcgtcatagacacc

SEQ ID NO: 33 - DNA, 20 bp, Artificial Sequence
  Description of Artificial Sequence: Synthetic primer
aggaatttgcatcagcgtgt

SEQ ID NO: 34 - DNA, 22 bp, Artificial Sequence
  Description of Artificial Sequence: Synthetic primer
aaaacaaattcatggttgctga

SEQ ID NO: 35 - DNA, 22 bp, Artificial Sequence
  Description of Artificial Sequence: Synthetic primer
tttacccatggtttaggaaagc

SEQ ID NO: 36 - DNA, 26 bp, Artificial Sequence
  Description of Artificial Sequence: Synthetic primer
ccaataatttgataaaggctattcac

SEQ ID NO: 37 - DNA, 20 bp, Artificial Sequence
  Description of Artificial Sequence: Synthetic primer
gactcttccctctgctctgg

SEQ ID NO: 38 - DNA, 23 bp, Artificial Sequence
  Description of Artificial Sequence: Synthetic primer
gaaaagaaagctagagactgcac

SEQ ID NO: 39 - DNA, 24 bp, Artificial Sequence
  Description of Artificial Sequence: Synthetic primer
tcctttagggaggatatctttcaa

SEQ ID NO: 40 - DNA, 21 bp, Artificial Sequence
  Description of Artificial Sequence: Synthetic primer
tcctcacttcccctaatgtcc

SEQ ID NO: 41 - DNA, 42 bp, Artificial Sequence
  Description of Artificial Sequence: Synthetic primer
acagtaatttctataattatcaaataatctcagctgaagaat

SEQ ID NO: 42 - DNA, 26 bp, Artificial Sequence
  Description of Artificial Sequence: Synthetic primer
catctctgatgtcacttttatgatac

SEQ ID NO: 43 - DNA, 30 bp, Artificial Sequence
  Description of Artificial Sequence: Synthetic primer
ttgtaattcacaacctgcataagttttcaa

SEQ ID NO: 44 - DNA, 22 bp, Artificial Sequence
  Description of Artificial Sequence: Synthetic primer
ctcgaattctcctcccaagatg

SEQ ID NO: 45 - DNA, 25 bp, Artificial Sequence
  Description of Artificial Sequence: Synthetic primer
ggctctacagtaatctggcttcaag

SEQ ID NO: 46 - DNA, 41 bp, Artificial Sequence
  Description of Artificial Sequence: Synthetic primer
gattttcaatatgtaaatgaaataacaatgtctgtagacaa

SEQ ID NO: 47 - DNA, 22 bp, Artificial Sequence
  Description of Artificial Sequence: Synthetic primer
ggaaatcttcctcaggcataca

SEQ ID NO: 48 - DNA, 17 bp, Artificial Sequence
  Description of Artificial Sequence: Synthetic primer
ggctgctgtggtttgca

SEQ ID NO: 49 - DNA, 19 bp, Artificial Sequence
  Description of Artificial Sequence: Synthetic primer
gcctgtaggccgctatgac

SEQ ID NO: 50 - DNA, 40 bp, Artificial Sequence
  Description of Artificial Sequence: Synthetic primer
actttccaaaagtaattttgacctaaaaatattaaccatt

SEQ ID NO: 51 - DNA, 25 bp, Artificial Sequence
  Description of Artificial Sequence: Synthetic primer
tgcattttccacattggcttctatg

SEQ ID NO: 52 - DNA, 21 bp, Artificial Sequence
  Description of Artificial Sequence: Synthetic primer
aaggacccagggctgaaaatc

SEQ ID NO: 53 - DNA, 30 bp, Artificial Sequence
  Description of Artificial Sequence: Synthetic primer
tcttccagaaagagattattgaacaaagca

SEQ ID NO: 54 - DNA, 25 bp, Artificial Sequence
  Description of Artificial Sequence: Synthetic primer
aaaggacagagtcatgttaaacaca

SEQ ID NO: 55 - skipped

SEQ ID NO: 56 - DNA, 18 bp, Artificial Sequence
  Description of Artificial Sequence: Synthetic primer
gcggcattcctggacttg

SEQ ID NO: 57 - DNA, 25 bp, Artificial Sequence
  Description of Artificial Sequence: Synthetic primer
tggaaacttactggtcatgattgct

SEQ ID NO: 58 - DNA, 40 bp, Artificial Sequence
  Description of Artificial Sequence: Synthetic primer
ttcctatactttatgaatcaagtaatttgatgtttactga

SEQ ID NO: 59 - DNA, 25 bp, Artificial Sequence
  Description of Artificial Sequence: Synthetic primer
atccttgactcttacctgcatcatg

SEQ ID NO: 60 - DNA, 29 bp, Artificial Sequence
  Description of Artificial Sequence: Synthetic primer
gctctattttatccttggtttaggaaagc

SEQ ID NO: 61 - DNA, 20 bp, Artificial Sequence
  Description of Artificial Sequence: Synthetic primer
tcaaaccatctcccccactt

SEQ ID NO: 62 - DNA, 25 bp, Artificial Sequence
  Description of Artificial Sequence: Synthetic primer
ctgactgtcaggaaaggacttagag

SEQ ID NO: 63 - DNA, 24 bp, Artificial Sequence
  Description of Artificial Sequence: Synthetic primer
tccctgtggcaataagagatagga

SEQ ID NO: 64 - DNA, 32 bp, Artificial Sequence
  Description of Artificial Sequence: Synthetic primer
tcaattttataccaacaaaaattccacaggtt

SEQ ID NO: 65 - DNA, 25 bp, Artificial Sequence
  Description of Artificial Sequence: Synthetic primer
atgtgagatactgctacaccctgta

SEQ ID NO: 66 - DNA, 30 bp, Artificial Sequence
  Description of Artificial Sequence: Synthetic primer
ccaatgcagatggaaaattgattcttaaca

SEQ ID NO: 67 - DNA, 31 bp, Artificial Sequence
  Description of Artificial Sequence: Synthetic primer
gttgcacagagaaactcaaaaggtaaaaata

SEQ ID NO: 68 - DNA, 24 bp, Artificial Sequence
  Description of Artificial Sequence: Synthetic primer
tgaatgggtcctgtgtgaaaatcc

SEQ ID NO: 69 - DNA, 19 bp, Artificial Sequence
  Description of Artificial Sequence: Synthetic primer
tcagcgtgtcctggatcct

SEQ ID NO: 70 - DNA, 21 bp, Artificial Sequence
  Description of Artificial Sequence: Synthetic primer
gatcctgcccctcatgtacag

SEQ ID NO: 71 - DNA, 26 bp, Artificial Sequence
  Description of Artificial Sequence: Synthetic primer
actgggtgttgacagattttctatcc

SEQ ID NO: 72 - DNA, 25 bp, Artificial Sequence
  Description of Artificial Sequence: Synthetic primer
caggagagagagaaaagcagctaaa

SEQ ID NO: 73 - DNA, 30 bp, Artificial Sequence
  Description of Artificial Sequence: Synthetic primer
gattcattaattgatgcctttatgggcttt

SEQ ID NO: 74 - DNA, 31 bp, Artificial Sequence
  Description of Artificial Sequence: Synthetic primer
cctttgatttatgctttattttacccatggt

SEQ ID NO: 75 - DNA, 27 bp, Artificial Sequence
  Description of Artificial Sequence: Synthetic primer
agcaaccatgaatttgttttctgaaca

SEQ ID NO: 76 - DNA, 26 bp, Artificial Sequence
  Description of Artificial Sequence: Synthetic primer
agaacagttcagcaaccatgaatttg

SEQ ID NO: 77 - DNA, 19 bp, Artificial Sequence
  Description of Artificial Sequence: Synthetic primer
cagcctgccaaaaatttca

SEQ ID NO: 78 - DNA, 22 bp, Artificial Sequence
  Description of Artificial Sequence: Synthetic primer
tctatgtcctttcttccccaaa

SEQ ID NO: 79 - DNA, 25 bp, Artificial Sequence
  Description of Artificial Sequence: Synthetic primer
tcctttcttccccaaatccataacc

SEQ ID NO: 80 - DNA, 25 bp, Artificial Sequence
  Description of Artificial Sequence: Synthetic primer
caaatccataacccccatgtagtca

SEQ ID NO: 81 - DNA, 20 bp, Artificial Sequence
  Description of Artificial Sequence: Synthetic primer
tcctttcttccccaaatcca

SEQ ID NO: 82 - DNA, 29 bp, Artificial Sequence
  Description of Artificial Sequence: Synthetic primer
aaggagatgtattaaagaagtggcattca

SEQ ID NO: 83 - DNA, 28 bp, Artificial Sequence
  Description of Artificial Sequence: Synthetic primer
acctgtccaattttgtttaaatgtgcat

SEQ ID NO: 84 - DNA, 28 bp, Artificial Sequence
  Description of Artificial Sequence: Synthetic primer
gtttctctcataagtcagtggttcttga

SEQ ID NO: 85 - DNA, 29 bp, Artificial Sequence
  Description of Artificial Sequence: Synthetic primer
aaaacttcttaccaaattctctcacaggt

SEQ ID NO: 86 - DNA, 18 bp, Artificial Sequence
  Description of Artificial Sequence: Synthetic primer
tcaggagctgtgcactgg

SEQ ID NO: 87 - DNA, 22 bp, Artificial Sequence
  Description of Artificial Sequence: Synthetic primer
ccccaaacatgcccatatccta

SEQ ID NO: 88 - DNA, 25 bp, Artificial Sequence
  Description of Artificial Sequence: Synthetic primer
gctgctgttaatgtgacctcttgat

SEQ ID NO: 89 - DNA, 21 bp, Artificial Sequence
  Description of Artificial Sequence: Synthetic primer
tgtggcatttgctgtgtccta

SEQ ID NO: 90 - DNA, 21 bp, Artificial Sequence
  Description of Artificial Sequence: Synthetic primer
tgtggcatttgctgtgtccta

SEQ ID NO: 91 - DNA, 24 bp, Artificial Sequence
  Description of Artificial Sequence: Synthetic primer
catggagaggatcaagaagttggt

SEQ ID NO: 92 - DNA, 25 bp, Artificial Sequence
  Description of Artificial Sequence: Synthetic primer
agtaactgtaggaggcatctacctc

SEQ ID NO: 93 - DNA, 25 bp, Artificial Sequence
  Description of Artificial Sequence: Synthetic primer
cttctctgtttgctactctcttcct

SEQ ID NO: 94 - skipped

SEQ ID NO: 95 - DNA, 25 bp, Artificial Sequence
  Description of Artificial Sequence: Synthetic primer
aagagaagcttgacattgtgcctat

SEQ ID NO: 96 - DNA, 33 bp, Artificial Sequence
  Description of Artificial Sequence: Synthetic primer
tccaccaaaataactattccagtcattaaaaga

SEQ ID NO: 97 - DNA, 23 bp, Artificial Sequence
  Description of Artificial Sequence: Synthetic primer
gtgtaactcagagccctatgcaa

SEQ ID NO: 98 - DNA, 21 bp, Artificial Sequence
  Description of Artificial Sequence: Synthetic primer
ctagcagcctgggaaaacagt

SEQ ID NO: 99 - DNA, 25 bp, Artificial Sequence
  Description of Artificial Sequence: Synthetic primer
gcaacaaagagaaccgggattttaa

SEQ ID NO: 100 - DNA, 30 bp, Artificial Sequence
  Description of Artificial Sequence: Synthetic primer
gattaaatgtagagttcaagtagccaaacc

SEQ ID NO: 101 - DNA, 34 bp, Artificial Sequence
  Description of Artificial Sequence: Synthetic primer
tcctctcacttctaaaacatttacctattactct

SEQ ID NO: 102 - DNA, 25 bp, Artificial Sequence
  Description of Artificial Sequence: Synthetic primer
gagagagagggagagagaaatgaca

SEQ ID NO: 103 - DNA, 22 bp, Artificial Sequence
  Description of Artificial Sequence: Synthetic primer
tgagtgtcgctacaacacgaaa

SEQ ID NO: 104 - DNA, 31 bp, Artificial Sequence
  Description of Artificial Sequence: Synthetic primer
tttgttagatttcttcctttatctgccaaga

SEQ ID NO: 105 - DNA, 22 bp, Artificial Sequence
  Description of Artificial Sequence: Synthetic primer
aaaaactctggcaatttgtatc

SEQ ID NO: 106 - DNA, 31 bp, Artificial Sequence
  Description of Artificial Sequence: Synthetic primer
gggagtgtttcctagaacatgaaaatttaag

SEQ ID NO: 107 - DNA, 28 bp, Artificial Sequence
  Description of Artificial Sequence: Synthetic primer
gaaatatgagttcagtgaaaaggcacag

SEQ ID NO: 108 - DNA, 24 bp, Artificial Sequence
  Description of Artificial Sequence: Synthetic primer
aagctcagctagagtctttcaagc

SEQ ID NO: 109 - DNA, 19 bp, Artificial Sequence
  Description of Artificial Sequence: Synthetic primer
cttccaggtgcaggagaca

SEQ ID NO: 110 - DNA, 25 bp, Artificial Sequence
  Description of Artificial Sequence: Synthetic primer
ccttagaagaggagtgagaggaaga

SEQ ID NO: 111 - DNA, 25 bp, Artificial Sequence
  Description of Artificial Sequence: Synthetic primer
tcatctggttttgtgggccattatt

SEQ ID NO: 112 - DNA, 25 bp, Artificial Sequence
  Description of Artificial Sequence: Synthetic primer
tgttactggagcatcaatccatgtt

SEQ ID NO: 113 - DNA, 24 bp, Artificial Sequence
  Description of Artificial Sequence: Synthetic primer
tccactttcctcttgccttctttc

SEQ ID NO: 114 - DNA, 18 bp, Artificial Sequence
  Description of Artificial Sequence: Synthetic primer
tgggcatgggaggagagt

SEQ ID NO: 115 - DNA, 21 bp, Artificial Sequence
  Description of Artificial Sequence: Synthetic primer
gggccctgccttataattgtt

SEQ ID NO: 116 - DNA, 32 bp, Artificial Sequence
  Description of Artificial Sequence: Synthetic primer
agagcaagaaaaacaaataaaatgcatacaga

SEQ ID NO: 117 - DNA, 25 bp, Artificial Sequence
  Description of Artificial Sequence: Synthetic primer
gcaatcagaaagtttgtaggtgtgt

SEQ ID NO: 118 - DNA, 25 bp, Artificial Sequence
  Description of Artificial Sequence: Synthetic primer
gtaaggcactgtgctaggtattgaa

SEQ ID NO: 119 - DNA, 32 bp, Artificial Sequence
  Description of Artificial Sequence: Synthetic primer
accttgcttctctatattctgaaatgtgatac

SEQ ID NO: 120 - DNA, 34 bp, Artificial Sequence
  Description of Artificial Sequence: Synthetic primer
tctaaaaataaactaatggttgatgaactagcct

SEQ ID NO: 121 - DNA, 20 bp, Artificial Sequence
  Description of Artificial Sequence: Synthetic primer
agaaggcccagctcacttca

SEQ ID NO: 122 - DNA, 31 bp, Artificial Sequence
  Description of Artificial Sequence: Synthetic primer
gtttttaaagaaatgtcaggtctaagatggg

SEQ ID NO: 123 - DNA, 28 bp, Artificial Sequence
  Description of Artificial Sequence: Synthetic primer
ggtaagttttataggctaggaggacatg

SEQ ID NO: 124 - DNA, 33 bp, Artificial Sequence
  Description of Artificial Sequence: Synthetic primer
tctttagtccattccatttcttttgagatttca

SEQ ID NO: 125 - DNA, 24 bp, Artificial Sequence
  Description of Artificial Sequence: Synthetic primer
ctaagccctcttcaacacttggta

SEQ ID NO: 126 - DNA, 19 bp, Artificial Sequence
  Description of Artificial Sequence: Synthetic primer
agccacccgggtttttgtt

SEQ ID NO: 127 - DNA, 37 bp, Artificial Sequence
  Description of Artificial Sequence: Synthetic primer
atttgacaaaaattatattgcacaagattattggaga

SEQ ID NO: 128 - DNA, 22 bp, Artificial Sequence
  Description of Artificial Sequence: Synthetic primer
tcaccaggaggtttccaaacac

SEQ ID NO: 129 - DNA, 22 bp, Artificial Sequence
  Description of Artificial Sequence: Synthetic primer
gataattcctccagcccatcgt

SEQ ID NO: 130 - DNA, 25 bp, Artificial Sequence
  Description of Artificial Sequence: Synthetic primer
cctcctatacagtttagggcatcag

SEQ ID NO: 131 - DNA, 24 bp, Artificial Sequence
  Description of Artificial Sequence: Synthetic primer
gaggatgattctgtcttgctacca

SEQ ID NO: 132 - DNA, 31 bp, Artificial Sequence
  Description of Artificial Sequence: Synthetic primer
ggcatcaattaatgaatcaatgctatatggt

SEQ ID NO: 133 - DNA, 27 bp, Artificial Sequence
  Description of Artificial Sequence: Synthetic primer
gctgagttataataagcacaccaacag

SEQ ID NO: 134 - DNA, 26 bp, Artificial Sequence
  Description of Artificial Sequence: Synthetic primer
caaattcatggttgctgaactgttct

SEQ ID NO: 135 - DNA, 29 bp, Artificial Sequence
  Description of Artificial Sequence: Synthetic primer
cgcttggtaattttaaaggtatcctgaac

SEQ ID NO: 136 - DNA, 35 bp, Artificial Sequence
  Description of Artificial Sequence: Synthetic primer
gaagagcaatttatttgctattcattcatagtctt

SEQ ID NO: 137 - DNA, 20 bp, Artificial Sequence
  Description of Artificial Sequence: Synthetic primer
caacagcctggtcaagatga

SEQ ID NO: 138 - DNA, 25 bp, Artificial Sequence
  Description of Artificial Sequence: Synthetic primer
tttcctgtagatcatgacagtttct

SEQ ID NO: 139 - DNA, 33 bp, Artificial Sequence
  Description of Artificial Sequence: Synthetic primer
cctgtagatcatgacagtttctcatactttatt

SEQ ID NO: 140 - DNA, 29 bp, Artificial Sequence
  Description of Artificial Sequence: Synthetic primer
tcctgtagatcatgacagtttctcatact

SEQ ID NO: 141 - DNA, 21 bp, Artificial Sequence
  Description of Artificial Sequence: Synthetic primer
cggatttcctcacttccccta

SEQ ID NO: 142 - DNA, 22 bp, Artificial Sequence
  Description of Artificial Sequence: Synthetic primer
tcccatcctttccctcctacaa

SEQ ID NO: 143 - DNA, 30 bp, Artificial Sequence
  Description of Artificial Sequence: Synthetic primer
tggtgttccttaaatattttccttggacaa

SEQ ID NO: 144 - DNA, 29 bp, Artificial Sequence
  Description of Artificial Sequence: Synthetic primer
gtgattgtgtgcatttttctagagaaaga

SEQ ID NO: 145 - DNA, 32 bp, Artificial Sequence
  Description of Artificial Sequence: Synthetic primer
cctgttattttttccattctctttttctcagt

SEQ ID NO: 146 - DNA, 21 bp, Artificial Sequence
  Description of Artificial Sequence: Synthetic primer
tgaaaaaccctgtgcgagtct

SEQ ID NO: 147 - DNA, 25 bp, Artificial Sequence
  Description of Artificial Sequence: Synthetic primer
ggtcctttgaccaagcaaggtaata

SEQ ID NO: 148 - DNA, 25 bp, Artificial Sequence
  Description of Artificial Sequence: Synthetic primer
ctgtatcgcttctgattcactctgt

SEQ ID NO: 149 - DNA, 20 bp, Artificial Sequence
  Description of Artificial Sequence: Synthetic primer
gagagcagcaggaagttggt

SEQ ID NO: 150 - DNA, 16 bp, Artificial Sequence
  Description of Artificial Sequence: Synthetic primer
gggccagggagagcag

SEQ ID NO: 151 - DNA, 25 bp, Artificial Sequence
  Description of Artificial Sequence: Synthetic primer
ctgggcaacatgatcgtaatgattt

SEQ ID NO: 152 - DNA, 25 bp, Artificial Sequence
  Description of Artificial Sequence: Synthetic primer
ctgttctgtgcttctaggttctgat

SEQ ID NO: 153 - DNA, 24 bp, Artificial Sequence
  Description of Artificial Sequence: Synthetic primer
agggaaggagcatgaagagtagag

SEQ ID NO: 154 - DNA, 25 bp, Artificial Sequence
  Description of Artificial Sequence: Synthetic primer
tctcctctgtctgattcactcaaga

SEQ ID NO: 155 - DNA, 18 bp, Artificial Sequence
  Description of Artificial Sequence: Synthetic primer
accccgaaggcaaatgct

SEQ ID NO: 156 - DNA, 22 bp, Artificial Sequence
  Description of Artificial Sequence: Synthetic primer
ctctgctcatagccttgtgtgt

SEQ ID NO: 157 - DNA, 25 bp, Artificial Sequence
  Description of Artificial Sequence: Synthetic primer
cccttagggtcttgtcttttggtta

SEQ ID NO: 158 - DNA, 21 bp, Artificial Sequence
  Description of Artificial Sequence: Synthetic primer
cctggcatatggagacaagct

SEQ ID NO: 159 - DNA, 24 bp, Artificial Sequence
  Description of Artificial Sequence: Synthetic primer
acagtcatggacccttttcttcac

SEQ ID NO: 160 - DNA, 25 bp, Artificial Sequence
  Description of Artificial Sequence: Synthetic primer
aatgtccagctccaggtaaaagttt

SEQ ID NO: 161 - DNA, 26 bp, Artificial Sequence
  Description of Artificial Sequence: Synthetic probe
gaaaaaggtaaaaaccatttccattt

SEQ ID NO: 162 - DNA, 23 bp, Artificial Sequence
  Description of Artificial Sequence: Synthetic probe
tgcacatgtgtgcaattaagagg

SEQ ID NO: 163 - DNA, 25 bp, Artificial Sequence
  Description of Artificial Sequence: Synthetic probe
aagagaaatacatcaacgcctagta

SEQ ID NO: 164 - DNA, 24 bp, Artificial Sequence
  Description of Artificial Sequence: Synthetic probe
aaattgcatatttcctttgaaaac

SEQ ID NO: 165 - DNA, 25 bp, Artificial Sequence
  Description of Artificial Sequence: Synthetic probe
caattataaaagctcaactgaacac

SEQ ID NO: 166 - DNA, 26 bp, Artificial Sequence
  Description of Artificial Sequence: Synthetic probe
ttggtttcttttgctttggttatttt

SEQ ID NO: 167 - DNA, 23 bp, Artificial Sequence
  Description of Artificial Sequence: Synthetic probe
gcagaatattcccgataaagttt

SEQ ID NO: 168 - DNA, 30 bp, Artificial Sequence
  Description of Artificial Sequence: Synthetic probe
cataatatttgatcagtatttctcaaaact

SEQ ID NO: 169 - DNA, 19 bp, Artificial Sequence
  Description of Artificial Sequence: Synthetic probe
cattaatcttcacattttt

SEQ ID NO: 170 - DNA, 15 bp, Artificial Sequence
  Description of Artificial Sequence: Synthetic probe
attggccacgttcct

SEQ ID NO: 171 - DNA, 18 bp, Artificial Sequence
  Description of Artificial Sequence: Synthetic probe
ttgaatagccgtggaatg

SEQ ID NO: 172 - DNA, 21 bp, Artificial Sequence
  Description of Artificial Sequence: Synthetic probe
ttaactgatctaaatttctca

SEQ ID NO: 173 - DNA, 19 bp, Artificial Sequence
  Description of Artificial Sequence: Synthetic probe
ccagagtctattcgcctgg

SEQ ID NO: 174 - DNA, 20 bp, Artificial Sequence
  Description of Artificial Sequence: Synthetic probe
aaggattaaagataattttt

SEQ ID NO: 175 - DNA, 23 bp, Artificial Sequence
  Description of Artificial Sequence: Synthetic probe
tctataagacataaaggaaggta

SEQ ID NO: 176 - DNA, 20 bp, Artificial Sequence
  Description of Artificial Sequence: Synthetic probe
ctatctgtaaaataaaacac

SEQ ID NO: 177 - DNA, 17 bp, Artificial Sequence
  Description of Artificial Sequence: Synthetic probe
tgggttatcccaaacag

SEQ ID NO: 178 - DNA, 18 bp, Artificial Sequence
  Description of Artificial Sequence: Synthetic probe
aaatcactgggtgtactt

SEQ ID NO: 179 - DNA, 16 bp, Artificial Sequence
  Description of Artificial Sequence: Synthetic probe
atctgccacgtaactt

SEQ ID NO: 180 - DNA, 16 bp, Artificial Sequence
  Description of Artificial Sequence: Synthetic probe
cttcacttcaaacaac

SEQ ID NO: 181 - DNA, 18 bp, Artificial Sequence
  Description of Artificial Sequence: Synthetic probe
cttagcagcaagtgaaaa

SEQ ID NO: 182 - DNA, 18 bp, Artificial Sequence
  Description of Artificial Sequence: Synthetic probe
ccaaaagcattcagggtt

SEQ ID NO: 183 - DNA, 23 bp, Artificial Sequence
  Description of Artificial Sequence: Synthetic probe
ttattttaagtggagatgtttta

SEQ ID NO: 184 - DNA, 18 bp, Artificial Sequence
  Description of Artificial Sequence: Synthetic probe
caaaagcatggaagacaa

SEQ ID NO: 185 - DNA, 18 bp, Artificial Sequence
  Description of Artificial Sequence: Synthetic probe
tcagtgcattcatagaag

SEQ ID NO: 186 - DNA, 13 bp, Artificial Sequence
  Description of Artificial Sequence: Synthetic probe
catgccgatttct

SEQ ID NO: 187 - DNA, 20 bp, Artificial Sequence
  Description of Artificial Sequence: Synthetic probe
acaacaaaaagaaatggcag

SEQ ID NO: 188 - DNA, 21 bp, Artificial Sequence
  Description of Artificial Sequence: Synthetic probe
tattgatgcttactatttaca

SEQ ID NO: 189 - DNA, 20 bp, Artificial Sequence
  Description of Artificial Sequence: Synthetic probe
atgctcttaatttgataaaa

SEQ ID NO: 190 - DNA, 19 bp, Artificial Sequence
  Description of Artificial Sequence: Synthetic probe
aaagccaaacactatgtac

SEQ ID NO: 191 - DNA, 16 bp, Artificial Sequence
  Description of Artificial Sequence: Synthetic probe
atgtacagcggtgctg

SEQ ID NO: 192 - DNA, 19 bp, Artificial Sequence
  Description of Artificial Sequence: Synthetic probe
cacaggtgtctatgacgat

SEQ ID NO: 193 - DNA, 23 bp, Artificial Sequence
  Description of Artificial Sequence: Synthetic probe
ttttctatctttttcgcctgtcg

SEQ ID NO: 194 - DNA, 17 bp, Artificial Sequence
  Description of Artificial Sequence: Synthetic probe
tcacagtgatagcattt

SEQ ID NO: 195 - DNA, 16 bp, Artificial Sequence
  Description of Artificial Sequence: Synthetic probe
ccctgcctatatttat

SEQ ID NO: 196 - DNA, 17 bp, Artificial Sequence
  Description of Artificial Sequence: Synthetic probe
ctgaccagttacaataa

SEQ ID NO: 197 - DNA, 17 bp, Artificial Sequence
  Description of Artificial Sequence: Synthetic probe
ttcgtctatccaactgc

SEQ ID NO: 198 - DNA, 17 bp, Artificial Sequence
  Description of Artificial Sequence: Synthetic probe
ttcgtctatccaactgc

SEQ ID NO: 199 - DNA, 18 bp, Artificial Sequence
  Description of Artificial Sequence: Synthetic probe
aaaatgtcagacgaaact

SEQ ID NO: 200 - DNA, 18 bp, Artificial Sequence
  Description of Artificial Sequence: Synthetic probe
actttatcgaggaatatt

SEQ ID NO: 201 - DNA, 21 bp, Artificial Sequence
  Description of Artificial Sequence: Synthetic probe
aaatccttcttaaaatttaag

SEQ ID NO: 202 - DNA, 22 bp, Artificial Sequence
  Description of Artificial Sequence: Synthetic probe
tcatatcaaaagagtacccatg

SEQ ID NO: 203 - DNA, 21 bp, Artificial Sequence
  Description of Artificial Sequence: Synthetic probe
atttcaatgtgactatgatct

SEQ ID NO: 204 - DNA, 22 bp, Artificial Sequence
  Description of Artificial Sequence: Synthetic probe
tgtataaatccataaggaagtg

SEQ ID NO: 205 - DNA, 20 bp, Artificial Sequence
  Description of Artificial Sequence: Synthetic probe
tatcccatctgttgtactag

SEQ ID NO: 206 - DNA, 18 bp, Artificial Sequence
  Description of Artificial Sequence: Synthetic probe
ttcccagaacctgttact

SEQ ID NO: 207 - DNA, 17 bp, Artificial Sequence
  Description of Artificial Sequence: Synthetic probe
ctctctacagaactaac

SEQ ID NO: 208 - DNA, 16 bp, Artificial Sequence
  Description of Artificial Sequence: Synthetic probe
cttcaaagcgcttcac

SEQ ID NO: 209 - DNA, 16 bp, Artificial Sequence
  Description of Artificial Sequence: Synthetic probe
cttcaaagcgcttcac

SEQ ID NO: 210 - DNA, 17 bp, Artificial Sequence
  Description of Artificial Sequence: Synthetic probe
atcgctcacttcaagca

SEQ ID NO: 211 - DNA, 16 bp, Artificial Sequence
  Description of Artificial Sequence: Synthetic probe
ccactacttctttccc

SEQ ID NO: 212 - DNA, 14 bp, Artificial Sequence
  Description of Artificial Sequence: Synthetic probe
ccctctgcctctcc

SEQ ID NO: 213 - DNA, 18 bp, Artificial Sequence
  Description of Artificial Sequence: Synthetic probe
cacagaaaagaaggccca

SEQ ID NO: 214 - DNA, 19 bp, Artificial Sequence
  Description of Artificial Sequence: Synthetic probe
cttctatgttggtcggtcc

SEQ ID NO: 215 - DNA, 18 bp, Artificial Sequence
  Description of Artificial Sequence: Synthetic probe
caacttgtcaaactgtca

SEQ ID NO: 216 - DNA, 22 bp, Artificial Sequence
  Description of Artificial Sequence: Synthetic probe
ctttggattcagtcctcagaga

SEQ ID NO: 217 - DNA, 17 bp, Artificial Sequence
  Description of Artificial Sequence: Synthetic probe
tttggaaccacgtcagc

SEQ ID NO: 218 - DNA, 20 bp, Artificial Sequence
  Description of Artificial Sequence: Synthetic probe
tttgaatgatgtgttgattt

SEQ ID NO: 219 - DNA, 16 bp, Artificial Sequence
  Description of Artificial Sequence: Synthetic probe
ttggaagggactttat

SEQ ID NO: 220 - DNA, 21 bp, Artificial Sequence
  Description of Artificial Sequence: Synthetic probe
tacattaatcttcatattttt

SEQ ID NO: 221 - DNA, 13 bp, Artificial Sequence
  Description of Artificial Sequence: Synthetic probe
tggccacattcct

SEQ ID NO: 222 - DNA, 17 bp, Artificial Sequence
  Description of Artificial Sequence: Synthetic probe
tgaatagccatggaatg

SEQ ID NO: 223 - DNA, 21 bp, Artificial Sequence
  Description of Artificial Sequence: Synthetic probe
ttaactgatctaaatctctca

SEQ ID NO: 224 - DNA, 19 bp, Artificial Sequence
  Description of Artificial Sequence: Synthetic probe
ccagagtctattcacctgg

SEQ ID NO: 225 - DNA, 20 bp, Artificial Sequence
  Description of Artificial Sequence: Synthetic probe
aaggattaaagatatttttt

SEQ ID NO: 226 - DNA, 18 bp, Artificial Sequence
  Description of Artificial Sequence: Synthetic probe
aagacataagggaaggta

SEQ ID NO: 227 - DNA, 20 bp, Artificial Sequence
  Description of Artificial Sequence: Synthetic probe
ctatctgtaaaatcaaacac

SEQ ID NO: 228 - DNA, 17 bp, Artificial Sequence
  Description of Artificial Sequence: Synthetic probe
tgggttatcccgaacag

SEQ ID NO: 229 - DNA, 20 bp, Artificial Sequence
  Description of Artificial Sequence: Synthetic probe
taaaatcactggatgtactt

SEQ ID NO: 230 - DNA, 17 bp, Artificial Sequence
  Description of Artificial Sequence: Synthetic probe
aatctgccatgtaactt

SEQ ID NO: 231 - DNA, 17 bp, Artificial Sequence
  Description of Artificial Sequence: Synthetic probe
ccttcacttctaacaac

SEQ ID NO: 232 - DNA, 17 bp, Artificial Sequence
  Description of Artificial Sequence: Synthetic probe
ttagcagcaaatgaaaa

SEQ ID NO: 233 - DNA, 16 bp, Artificial Sequence
  Description of Artificial Sequence: Synthetic probe
aaaagcatgcagggtt

SEQ ID NO: 234 - DNA, 18 bp, Artificial Sequence
  Description of Artificial Sequence: Synthetic probe
ttaagtggagctgtttta

SEQ ID NO: 235 - DNA, 15 bp, Artificial Sequence
  Description of Artificial Sequence: Synthetic probe
aagcatggaaggcaa

SEQ ID NO: 236 - DNA, 17 bp, Artificial Sequence
  Description of Artificial Sequence: Synthetic probe
cagtgcattcgtagaag

SEQ ID NO: 237 - DNA, 13 bp, Artificial Sequence
  Description of Artificial Sequence: Synthetic probe
catgccaatttct

SEQ ID NO: 238 - DNA, 19 bp, Artificial Sequence
  Description of Artificial Sequence: Synthetic probe
caacaaaaagagatggcag

SEQ ID NO: 239 - DNA, 19 bp, Artificial Sequence
  Description of Artificial Sequence: Synthetic probe
ttgatgcttactgtttaca

SEQ ID NO: 240 - DNA, 20 bp, Artificial Sequence
  Description of Artificial Sequence: Synthetic probe
atgctcttaatttcataaaa

SEQ ID NO: 241 - DNA, 18 bp, Artificial Sequence
  Description of Artificial Sequence: Synthetic probe
aagccaaacactgtgtac

SEQ ID NO: 242 - DNA, 16 bp, Artificial Sequence
  Description of Artificial Sequence: Synthetic probe
atgtacagcagtgctg

SEQ ID NO: 243 - DNA, 17 bp, Artificial Sequence
  Description of Artificial Sequence: Synthetic probe
caggtgtctgtgacgat

SEQ ID NO: 244 - DNA, 22 bp, Artificial Sequence
  Description of Artificial Sequence: Synthetic probe
tttctatctttttcacctgtcg

SEQ ID NO: 245 - DNA, 15 bp, Artificial Sequence
  Description of Artificial Sequence: Synthetic probe
acagtggtagcattt

SEQ ID NO: 246 - DNA, 15 bp, Artificial Sequence
  Description of Artificial Sequence: Synthetic probe
cctgcctgtatttat

SEQ ID NO: 247 - DNA, 17 bp, Artificial Sequence
  Description of Artificial Sequence: Synthetic probe
ctgaccagttataataa

SEQ ID NO: 248 - DNA, 17 bp, Artificial Sequence
  Description of Artificial Sequence: Synthetic probe
ttcgtctatacaactgc

SEQ ID NO: 249 - DNA, 17 bp, Artificial Sequence
  Description of Artificial Sequence: Synthetic probe
ttcgtctatacaactgc

SEQ ID NO: 250 - DNA, 15 bp, Artificial Sequence
  Description of Artificial Sequence: Synthetic probe
atgtcagacaaaact

SEQ ID NO: 251 - DNA, 16 bp, Artificial Sequence
  Description of Artificial Sequence: Synthetic probe
ctttatcgggaatatt

SEQ ID NO: 252 - DNA, 20 bp, Artificial Sequence
  Description of Artificial Sequence: Synthetic probe
aatccttcttaaactttaag

SEQ ID NO: 253 - DNA, 22 bp, Artificial Sequence
  Description of Artificial Sequence: Synthetic probe
tcatatcaaaagagtccccatg

SEQ ID NO: 254 - DNA, 18 bp, Artificial Sequence
  Description of Artificial Sequence: Synthetic probe
tcaatgtgactgtgatct

SEQ ID NO: 255 - DNA, 20 bp, Artificial Sequence
  Description of Artificial Sequence: Synthetic probe
tataaatccataagaaagtg

SEQ ID NO: 256 - DNA, 21 bp, Artificial Sequence
  Description of Artificial Sequence: Synthetic probe
ctatcccatctattgtactag

SEQ ID NO: 257 - DNA, 19 bp, Artificial Sequence
  Description of Artificial Sequence: Synthetic probe
attcccagaacttgttact

SEQ ID NO: 258 - DNA, 15 bp, Artificial Sequence
  Description of Artificial Sequence: Synthetic probe
ctctaccgaactaac

SEQ ID NO: 259 - DNA, 16 bp, Artificial Sequence
  Description of Artificial Sequence: Synthetic probe
cttcaaagcacttcac

SEQ ID NO: 260 - DNA, 16 bp, Artificial Sequence
  Description of Artificial Sequence: Synthetic probe
cttcaaagcacttcac

SEQ ID NO: 261 - DNA, 16 bp, Artificial Sequence
  Description of Artificial Sequence: Synthetic probe
tcgctcactttaagca

SEQ ID NO: 262 - DNA, 15 bp, Artificial Sequence
  Description of Artificial Sequence: Synthetic probe
cactacttgtttccc

SEQ ID NO: 263 - DNA, 14 bp, Artificial Sequence
  Description of Artificial Sequence: Synthetic probe
ccctctgtctctcc

SEQ ID NO: 264 - DNA, 17 bp, Artificial Sequence
  Description of Artificial Sequence: Synthetic probe
acagaaaagcaggccca

SEQ ID NO: 265 - DNA, 21 bp, Artificial Sequence
  Description of Artificial Sequence: Synthetic probe
tacttctatgttagtcggtcc

SEQ ID NO: 266 - DNA, 17 bp, Artificial Sequence
  Description of Artificial Sequence: Synthetic probe
aacttgtcaagctgtca

SEQ ID NO: 267 - DNA, 20 bp, Artificial Sequence
  Description of Artificial Sequence: Synthetic probe
ttggattcagtccccagaga

SEQ ID NO: 268 - DNA, 18 bp, Artificial Sequence
  Description of Artificial Sequence: Synthetic probe
ctttggaaccatgtcagc

SEQ ID NO: 269 - DNA, 21 bp, Artificial Sequence
  Description of Artificial Sequence: Synthetic probe
ctttgaatgatgtattgattt

SEQ ID NO: 270 - DNA, 18 bp, Artificial Sequence
  Description of Artificial Sequence: Synthetic probe
ttttggaaggaactttat

SEQ ID NO: 271 - DNA, 107 bp, Homo sapiens
tgtagtcatgagaaaaatgtcagacgaaactttatcgggaatattctgcataatatttgatcagtatttctcaaaactgtcagtcatcaaaaataaagtatgagaaa

SEQ ID NO: 272 - DNA, 76 bp, Mus musculus
tgtagtagtctacagtctacagaacaagagaaaaattgttgcttgctatacacctgacacggaactgacacccaaa